(19) United States
(12) Patent Application Publication
(10) Pub. No.: US 2026/0015581 A1
(43) Pub. Date: Jan. 15, 2026

(54) METHODS FOR THE PRODUCTION OF RETINAL CELLS

(71) Applicant: MIAMI UNIVERSITY, OXFORD, OH (US)

(72) Inventors: PHUONG T. LAM, OXFORD, OH (US); KATIA DEL RIO-TSONIS, DAYTON, OH (US); MICHAEL L. ROBINSON, LIBERTY, IN (US)

(21) Appl. No.: 19/231,161

(22) Filed: Jun. 6, 2025

Related U.S. Application Data
(62) Division of application No. 16/390,766, filed on Apr. 22, 2019.
(60) Provisional application No. 62/660,590, filed on Apr. 20, 2018.

(51) Int. Cl. C12N 5/079; C12N 15/85; C12N 15/87

(52) U.S. Cl. CPC C12N 5/0621; C12N 15/85; C12N 15/87; C12N 2506/45

(57) ABSTRACT

The general inventive concepts contemplate methods and compositions for modifying certain cell lines to facilitate the production of retinal tissue from human induced pluripotent stem cells. Human induced pluripotent stem cells can be cultured to develop into retinal organoids, among other cells types. The retinal organoids play a role in modeling both human retinal development and retinal disease. The methods and compositions discussed herein provide means to measure and monitor human induced pluripotent stem cells during development.

### RELATED APPLICATION

This application is a division of U.S. Non-Provisional application Ser. No. 16/390,766, filed Apr. 22, 2019 and claims the benefit of U.S. Provisional Application No. 62/660,590, filed Apr. 20, 2018, the content of which is incorporated by reference herein in its entirety.

### SEQUENCE LISTING

The instant application contains a Sequence Listing which has been filed electronically in ASCII format and is hereby incorporated by reference in its entirety. Said ASCII copy, created on Jan. 2, 2020, is named 27876_04021_SL.txt and is 58,223 bytes in size.

### FIELD

The general inventive concepts relate to the field of medical research and more particularly to methods for the production of cells for use in medical research.

### BACKGROUND

There are a variety of obstacles present for researchers interested in developing therapies to treat visual diseases and/or for those interested in researching general retinal development. One major obstacle is the availability of tissue on which test/observe. Thus, there exists a need for methods to produce tissues for research into various visual-related conditions and diseases.

### SUMMARY

The general inventive concepts relate to and contemplate methods and compositions for producing retinal cells. The general inventive concepts are based in large part on three unique genetic insertions of fluorescent reporter genes that specifically label neural retina cell types. The particular genetic insertions of fluorescent reporter genes allows for accurate tracking of the differentiation/development of human induced pluripotent stem cells (hiPSCs) through normal retina development while exhibiting appropriate fluorescent protein expression consistent with the onset of retina progenitors (NRPs), retinal ganglion cells (RGCs), and photoreceptors (PRs), respectively.

As mentioned previously, a need exists for increasing the availability of retinal tissues for research and testing of therapies to treat various eye-related conditions and diseases. One potential avenue for producing such tissues is stem cells, more particularly hiPSCs.

Accordingly, the general inventive concepts relate to and contemplate a transgenic cell line, including triple targeted hiPSCs. Such triple targeted hiPSCs may take the form of retina organoids.

In an exemplary embodiment, the general inventive concepts contemplate a method for the production of retinal tissue, including retina organoids. The method comprises providing hiPSCs, editing at least one neural retina-specific gene. In certain embodiments, the method comprises determining the development of the tissue by monitoring an output resulting from expression of the at least one gene. In certain embodiments, determining comprises applying one or more predetermined wavelengths of light.

In an exemplary embodiment, the general inventive concepts contemplate a vector for editing at least one neural retina-specific gene. In certain embodiments, the vector is directed to label a particular neural retina cell type. In certain embodiments, the vector is targeted at a retina-specific gene. In certain embodiments, the retina-specific gene is a gene associated with a particular stage of retina development.

In certain exemplary embodiments, the general inventive concepts are directed to a transfection kit comprising an expression vector comprising a first nucleic acid molecule encoding a first fluorescent protein.

Numerous other aspects, advantages, and/or features of the general inventive concepts will become more readily apparent from the following detailed description of exemplary embodiments and from the accompanying drawings being submitted herewith.

### BRIEF DESCRIPTION OF THE DRAWINGS

The general inventive concepts, as well as embodiments and advantages thereof, are described below in greater detail, by way of example, with reference to the drawings in which:

FIG. 1 shows the PCR strategy used to identify appropriately targeted clones is described in FIG. 1

FIG. 2 shows a CRISPR/Cas9 based strategy to replace the stop codon of the targeted locus with a P2A (e.g., SEQ ID NO:2)/Fluorescent reporter fusion gene by homology direct repair.

FIG. 3 shows human retinal organoids produced from triple-targeted hiPSCs according to the general inventive concepts.

FIG. 4 shows the generation of the hiPSC triple transgenic line.

FIG. 5 shows the creation of the neural retina Progenitor/Retinal Ganglion Cell/Photoreceptor (PGP1) Reporter hiPSC Line by CRISPR/Cas9 Genome Editing. (A) Schematic illustration of the generalized CRISPR/Cas9-mediated insertion strategy. CRISPR/Cas9 mediated the replacement of the endogenous STOP codon of VSX2 (B), BRN3b (C), and RCVRN (D) loci with P2A: Cerulean, P2A: eGFP, and P2A: mCherry by homologous recombination in WT hiPSCs. Following nucleofection and triple antibiotic selection for Puromycin (PURO), Blasticidin (BLAST), and G418 (NEO) the resistant clones were screened by PCR with primer sets. (B) FW1/RV1 (forward primer (FW) located outside VSX2 5′HA and reverse primer (RV) located inside Cerulean) with the expected band size of 2.1 kb, and FW2/RV2 (inside Puromycin to outside VSX2 3′HA) with the expected band size of 1.9 kb. (C) FW3/RV3 (outside BRN3b 5′HA to inside membrane tagged enhanced GFP) with the expected band size of 1.7 kb; and FW4/RV4 (inside Blasticidin to outside BRN3b 3′HA) with the expected band size of 1.5 kb. (D) FW5/RV5 (outside RCVRN 5′HA to inside mCherry) with the expected band size of 1.2 kb; and FW6/RV6 (inside NEO to outside RCVRN 3′HA) with the expected band size of 1.5 kb. The WT hiPSC was used as control where no bands were seen. All the positive PCR bands were verified by sequencing. The original gels that were cropped for clarity in this figure (with white spaces between non-adjacent lanes) can be seen in their entirety in FIG. 15.

FIG. 6 show results of experiments to determine the zygosity of the PGP1 line. In each case, a three-primer PCR strategy determined whether the PGP1 clone was homozygous or heterozygous at the targeted loci. For the VSX2 locus, primer FW1 is located outside the VSX2 5′HA, RV1 is located inside Cerulean, and RV2 is located outside the VSX2 3′HA. (A) The un-edited VSX2 allele (FW1/RV2) should generate a 2.6 kb band, (A′) while the edited VSX2 allele (FW1/RV1) should generate a 2.1 kb band. (A″) A gel image showed bands of 2.6 kb and a 2.1 kb for the PGP1 line while the WT hiPSC showed a 2.6 kb band indicating that PGP1 is heterozygous at the VSX2 locus. For the BRN3b locus, FW3 is located outside the BRN3b 5′HA, RV3 is located inside the eGFP, and RV4 is located outside the BRN3b 3′HA. (B) The un-edited BRN3b allele (FW3/RV4) should generate a 2.4 kb band, (B′) while the edited BRN3b allele (FW3/RV3) should generate a band of 1.7 kb. (B″) A gel image showed bands of 2.4 kb and a 1.7 kb for the PGP1 line while the WT hiPSC showed only a 2.4 kb band, showing that PGP1 is heterozygous at the BRN3b locus. For the RCVRN locus, FW5 is located outside the RCVRN 5′HA, RV5 is located inside mCherry, and RV6 is located outside the RCVRN 3′HA. (C) The un-edited RCVRN allele (FW5/RV6) should generate a 2.2 kb band, (C′) while the edited RCVRN allele (FW5/RV5) should generate a 1.2 kb band. (C″) A gel image showed bands of 2.2 kb and a 1.2 kb for the PGP1 line while the WT hiPSC showed only a 2.2 kb band, indicating that PGP1 is heterozygous at the RCVRN locus. The original gels that were cropped for clarity in this figure (with white spaces between non-adjacent lanes) can be seen in their entirety in FIG. 16.

FIG. 7 shows experiments to determine lack of indel mutations in the non-targeted alleles of PGP1. Sequence analysis of the WT alleles of VSX2 (A), BRN3b (B) and RCVRN (C) failed to detect any Cas9-mediated indel mutations in PGP1. Orange arrows represent the sgRNA sequence, the endogenous stop codons are shaded in pink and coding sequences are represented by green rectangles.

FIG. 8 shows cerulean positive retina progenitors appear before eGFP or mCherry positive cells during PGP1 retinal organoid differentiation. After 20 days of differentiation, retinal domains (A) first express Cerulean (blue) (B), but not eGFP (C) or mCherry (D). The composite of the bright field and VSX2/Cerulean (E). Magnification bar 20 μm.

FIG. 9 shows the brightfield view of three dimension retinal organoids at D55 of differentiation. Free-floating organoids have variable size but all maintain a three dimensional shape with characteristic spherical structure with a distinct thick exterior and hollower interior. Scale bar 40 μM

FIG. 10 shows the functional analysis of the fluorescent reporters in the PGP1 line via retinal organoid formation. Single three-dimensional retinal organoids derived from the PGP1 hiPSC line were visualized by fluorescent microscopy at D55 (A, B, C, D), D95 (E, F, G, H) and D135 (I, J, K, L) of differentiation. Organoids were visualized to excite Cerulean, driven by the VSX2 promoter (A, E, I), eGFP driven by the BRN3b promoter (B, F J) and mCherry driven by the RCVRN promoter (C, G, K). Composite images represent the merger of all three fluorescent signals (D, H, L). The eGFP signal was targeted to the cell membrane by a GAP43 tag to allow for visualization of ganglion cell axons (white arrows in B, D). As organoids matured from D55 to D135, the number of cells expressing mCherry dramatically increased (compare C and K) and the eGFP positive cells populated the interior of the organoid while the mCherry positive cells occupy the organoid periphery (see J and K). Magnification Bar 100 μm.

FIG. 11A: Confirmation of PGP1-derived retinal organoids corresponded to the appropriately targeted cell types. At D55 (A-F, J-L) and D135 (G-I), the retinal organoids were dissociated into single cells, and used for FACS analysis. At D55, (A) the Cerulean positive population was sorted from the Cerulean negative population. RT-qPCR analysis revealed that the Cerulean positive cells expressed significantly more Cerulean mRNA (B) and VSX2 mRNA (C) than the Cerulean negative cells. Also at D55, (D) the eGFP positive population was sorted from the GFP negative population. RT-qPCR analysis demonstrated that the eGFP positive cells expressed significantly more eGFP mRNA (E) and Brn3b mRNA (F) than the eGFP negative cells. At D135, (G) the mCherry positive population was sorted from the mCherry negative population. RT-qPCR analysis revealed that the mCherry positive cells expressed significantly more mCherry mRNA (H) and RCVRN mRNA (I) than mCherry negative cells. As a negative control, retinal organoids (D55) derived from wild-type hiPSC cells were run through the FACS and analyzed with the gates used for Cerulean (J), eGFP (K) and mCherry (L) with no cells occupying those gates. All RT-qPCR data was normalized to GAPDH expression. Error bars represent standard error of the mean (SE).

FIG. 11B is continuation of FIG. 11A showing additional test results.

FIG. 12: Retinal development is recapitulated in the differentiating PGP1 hiPSC-derived retinal organoids. Undifferentiated PGP1 hiPSCs day 0 (D0, blue) and differentiated PGP1 retinal organoids at day 55 (D55, orange) were used to measure mRNA levels of various markers via RT-qPCR normalized to GAPDH. (A) Expression of stem cell markers OCT4, NANOG; (B) retinal progenitors and neuronal markers—PAX6, SIX3, LHX2, VSX2; (C) ganglion cell markers—BRN3a, BRN3b; (D) photoreceptor marker-RCVRN; and (E) retinal pigmented epithelium markers—MITF, BEST1. As organoid differentiation progressed from D0 to D55 all stem cell markers significantly decreased and markers of retina and RPE differentiation significantly increased. Error bars represent standard error of the mean (SE).

FIG. 13: retinal organoids differentiated from PGP1 line contain all major retinal cell types. At D55 of retinal organoid differentiation, the organoids were analyzed by immunohistochemistry using antibodies for the eye-field precursor marker RX (A), neuronal markers: PAX6 (B), SIX3 (C), the neural retina progenitor cell marker VSX2 (D), the proliferation marker MCM2 (E), and the retinal ganglion cell marker BRN3b (F). At D70 of the retinal organoid differentiation, the organoids contain the amacrine cell marker AP-2α (G) and the photoreceptor marker RCVRN (H). The organoids contain the horizontal cell marker Prox-1 (I) at D95, and the Müller glia cell marker CRALBP (J) at D163. Also at D163, the organoids contain the bipolar cell marker VSX2+/MCM2− (K, L). (L) is an enlarged view of the boxed area in (K) where VSX2+/MCM2+ (red arrows) cells represent neural retina progenitors and the VSX2+/MCM2− (white arrows) cells represent bipolar cells at D163. Dotted lines emphasize that the major expression domains of Brn3b (F), AP2α (G) and Prox1 (I) appear on the interior of the organoids while RCVRN (H) is predominantly on the organoid periphery. Magnification Bar 100 μm (A-K), 25 μm (L).

FIG. 14A: Establishing FACS Gates Using Transiently Transfected HEK293 Cells. Wild-type HEK293 cells (A-D) or HEK293 cells transiently transfected with expression plasmids for Cerulean (E-H), eGFP (I-L), or mCherry (M-P) were dissociated into single cell populations (A, E, I, M) and Gates were established for each fluorescent protein based on parameters that would lead to capturing the appropriate fluorescent protein expressing cells without capturing any wild-type cells. Applicants confirmed that the captured cells in the Cerulean positive gate (F) the eGFP positive gate (K) and the mCherry positive gate (P) expressed the appropriate fluorescent protein when sorted and cultured.

FIG. 14B is continuation of FIG. 14A showing additional test results.

FIG. 15: The original ethidium bromide stained PCR gels used to support FIG. 5. PCR reactions using genomic DNA as template with the primers indicated above each lane. The expected band sizes for each targeted allele are shown above each lane. The template DNA for the gel on the left came from the PGP1 clone while the template for the gel on the right was from a wild-type hiPSC clone. MW indicates a DNA size ladder run on each gel.

FIG. 16: The original ethidium bromide stained PCR gel that was cropped for clarity in FIG. 6. The PGP1 cell line and wild-type hiPSCs (WT) provided the genomic DNA template for a three primer PCR strategy to detect the wild-type and targeted alleles for the VSX2, BRN3b and RCVRN loci. The primers used for each reaction are indicated above the relevant lanes. MW indicates DNA size ladders run in duplicate on the gel.

FIG. 17 shows confirmation that fluorescent protein expression in PGP1-derived retinal cup organoids does not survive fixation and frozen sectioning. Sections of PGP1 hiPSC-derived retinal organoids were prepared after fixation in 4% paraformaldehyde, overnight incubation in 30% sucrose at 4° C., and embedding in OCT compound. Organoid sections from D55 (A-C), D75 (D-F), D95 (G-I), and D166 (J-L) of differentiation were visualized following DAPI staining on the blue DAPI filter (A, D, G, J), the green FITC filter for GFP (B, E, H, K) and the red Texas Red filter for mCherry (C, F, I, L). No green or red signals consistent with eGFP or mCherry were detected in organoids of any age. Using an LSM 800 confocal system, an unstained organoid section from D55 was visualized for cerulean expression (Ex.433 nm, Em.475) (M), eGFP (Ex.493 nm, Em. 517) (N), and mCherry (Ex.577, Em.603) (O). The insert (M′) showed the D55 RC section in brightfield. Ex is excitation wavelength and Em is Emission wavelength. Magnification bar for (M′) is 50 μm, and 100 μm for all other images.

FIG. 18 shows additional controls for PGP1-derived retinal cup organoids. To ensure the signals from immunofluorescent staining experiments are specific for the intended antigens, the retina cup organoids were stained for DAPI and secondary antibody only (A-L). The staining for DAPI, and the secondary antibodies Donkey anti Sheep Alexa Fluor 488, and Donkey anti Sheep Alexa Fluor 546 were done for sections from organoids at D55 (A-C), D75 (D-F), D95 (G-I), and D166 (J-L) of differentiation. To ensure that the DAPI signal did not represent the VSX2-Cerulean signal, D55 organoid sections were stained with DAPI, and a sheep anti-VSX2 primary antibody and secondary anti sheep Alexa Fluor 488 antibody (M-O). D95 organoid sections were stained with DAPI, and a sheep anti-VSX2 primary antibody and a secondary antisheep Alexa Fluor 546 antibody (P-R). Note that only a subset of the DAPI signals in (M) and (P) are VSX2 positive (N, R). Images in the first column (A, D, G, J, M, P) were photographed with a DAPI filter, while images in the middle (B, E, H, K, N, Q) were photographed with a FITC filter and the last column (C, F, I, L, O, R) were photographed with a Texas Red filter. Magnification Bar. 50 μm, applies to all images.

FIG. 19 shows PGP1-derived RPE treated with the indicated compounds at the initiation of the experiment (D0) or for 10 (D10) and 15 (D15) days. Images were taken under brightfield and darkfield for blue fluorescence and shown as a composite image. Other than the absence of the indicated chemical, the control cultures were treated identically.

FIG. 20A shows representative images from the RPE to NR candidates from the Selleck Chemicals Epigenetics library. PGP1-derived RPE cells are shown at the initiation of the experiment D0 or after 5 (D5) and 10 (D10) days. Images are shown as a composite of brightfield illumination (to show RPE pigmentation and darkfield fluorescence to show the blue (Cerulean) expression. The concentration of the compounds is: U0126: 39 μM, SC 79:110 μM, and KU 0063794:3 μM. The negative control cultures (top row) were not treated with the indicated compounds, but were otherwise treated identically (with the same media). All of these treatments have been repeated three times.

FIG. 20B is continuation of FIG. 20A showing additional test results.

FIG. 21 shows the results of treatment with BI 847325 at the initiation of the experiment (D0), and after 5 (D5) and 10 (D10) days of treatment. The top row shows a composite of brightfield illumination to visualize pigmentation and darkfield illumination to visualize blue (Cerulean) expression and green (eGFP). The same images are shown in the lower row with only the darkfield views to visualize green (eGFP) expression.

FIG. 22 shows the DNA sequence for the VSX2-P2A-Cerulean repair template for the PGP1 cell line (e.g., SEQ ID NOs: 1-19).

FIG. 23 shows the DNA sequence for the Brn3b-P2A-eGFP repair construct for PGP1 cell line (e.g., SEQ ID NOs: 5 and 20-37).

FIG. 24 shows the sequence for the repair template for RCVRN-P2A-mCherry for the PGP1 cell line (e.g., SEQ ID NOs: 5-6, 10-12, and 38-56).

### DETAILED DESCRIPTION

While the general inventive concepts are susceptible of embodiment in many different forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

The materials, systems, and methods described herein are intended to be used to provide retinal tissues with improved characteristics as well as vectors for modifying hiPSCs.

Stem cells provide multiple avenues to address irreversible vision loss associated with retinal cell death caused by trauma or by diseases including Age-Related Macular Degeneration (AMD) and glaucoma. AMD and glaucoma alone leave millions of people blind and cost billions of dollars in social welfare and lost productivity. Current treatments for these diseases may slow the progression of retinal degeneration, but no available treatments restore lost retinal tissue. Recent advancements in 3D-culture have led to the development of retinal organoids, that consist of all major retinal cell types from human induced pluripotent stem cells (hiPSCs)³. This capability means that these hiPSC-derived organoids can experimentally model normal human retina development as well as onset and progression of retinal disease. Additionally, these organoids can provide a platform to screen new drugs for the treatment or the cure of retinal disease. Finally, hiPSCs provide the ability to make unlimited numbers of specific retinal neurons for potential transplantation therapies.

There is an unmet need for tools to monitor the appearance of specific cell types without having to interrupt the normal developmental process. Having multiple cell type reporters inserted in the same genome will permit specific cell sorting and allow for the optimization of protocols that enrich for the development of one retinal cell type over another. For example, one might need a large number of photoreceptors or ganglion cells to screen for drugs to treat AMD or glaucoma, respectively. To address these limitations, Applicants utilized CRISPR/Cas9 genome editing to create a hiPSC retina reporter line to monitor the development of neural retina progenitor (NRP), retinal ganglion (RGC), and photoreceptor (PR) cells.

Several important considerations went into choosing a strategy to target NRPs, RGCs and PRs without damaging the ability of the engineered hiPSC line to undergo normal retinal development. VSX2 encodes a transcription factor that marks NRPs before they differentiate into mature retinal cell types. In the mature retina, only post-mitotic bipolar cells express VSX2 (e.g., SEQ ID NO: 1). RGCs represent the first mature retinal neuron cell type to develop and most RGCs express the BRN3b (e.g., SEQ ID NO: 20) transcription factor. PRs (rods and cones) express RCVRN (e.g., SEQ ID NO: 38), a calcium binding protein important in the recovery phase of visual excitation. The cell-type-specific expression pattern of these genes made them appropriate endogenous targets for the insertion of fluorescent reporter genes. Since these three genes all play an important role in retina development and/or function, the ideal reporter hiPSC line will retain the function of both alleles of all three of these genes. Therefore, viral P2A peptides were utilized to create fusion genes between the target and the fluorescent reporter that would self-cleave upon translation.

The value of a multiple-targeted retina reporter line depends on its ability to differentiate faithfully into all retinal cell types in a stereotypical fashion while simultaneously providing visible readout without the need to add dyes or to stop the developmental process. Here, a P2A: Cerulean reporter was inserted into the VSX2 locus, a P2A: eGFP reporter into the BRN3b locus and a P2A: mCherry reporter into the RCVRN locus. In each case, CRISPR/Cas9 genome editing was used to replace the stop codon of the endogenous gene with the P2A reporter. Validation of the resultant triple transgenic hiPSC (PGP1) line came from the ability of these cells to generate retinal organoids with the appropriate expression of each fluorescent reporter gene.

hiPSCs can be differentiated into virtually any mature human cell type, but the protocols to achieve optimum numbers of particular human cell types and tissues often remain inefficient for the generation of human tissue for clinical transplantation. The triple targeted hiPSC line according to the general inventive concepts will make it possible to evaluate the success of human retina differentiation protocols in real time without having to fix or destroy the tissue. Human retina organoids made from these hiPSCs will also make it possible to evaluate the effects of various drug treatment regimens on specific retinal cell types. These hiPSCs could also be used as research tools to follow retinal ganglion cell axon guidance, and retina regeneration therapies.

Accordingly, the general inventive concepts relate to a new cell line with validation of expression of each of three fluorescent proteins in retinal organoid cultures (also referred to as the triple-targeted hiPSC line). Applicants have also verified that each gene is targeted on only one of the two alleles and that the non-targeted allele is free from CRISPR/Cas9-generated indel mutations. At present, Applicants are unaware of any hiPSC line that expresses more than one cell-type specific reporter gene for retinal development. The triple targeted hiPSC line has three different reporters making it possible to simultaneously follow immature retinal progenitor cells early in development and retinal ganglion cells, photoreceptors and bipolar neurons in the mature retina in real time. This makes the current cell line more versatile and amenable for studies of how different retinal neurons connect and interact with each other through development and under different drug treatment regimens.

Retinal organoid formation from the cell line described according to the general inventive concepts (also called PGP1 herein) demonstrated the ability of the edited cells to undergo normal retina development while exhibiting appropriate fluorescent protein expression consistent with the onset of NRPs, RGCs, and PRs. Organoids produced from the PGP1 line expressed transcripts consistent with the development of all major retinal cell types. The PGP1 line offers a powerful new tool to study retinal development, retinal reprogramming, and therapeutic drug screening

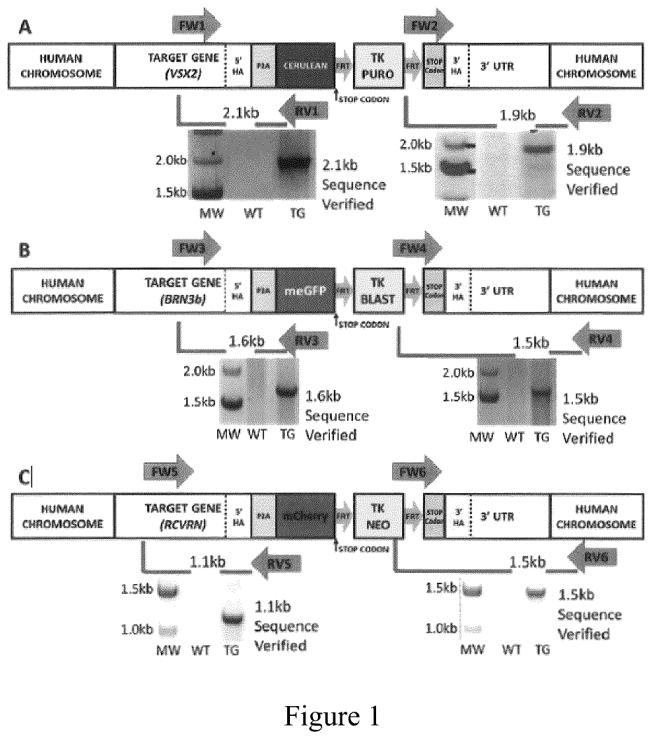

The triple-targeted hiPSC line addresses the need to follow retina cell differentiation in real time with a reporter that can be easily visualized in living cells. This hiPSC line was created using CRISPR/Cas9-mediated gene editing with homologous recombination. Multiple triple targeted hiPSC clones were identified by PCR following nucleofection of a sgRNA, a homologous recombination template and a Cas9 expression vector. The mCherry RCVRN hiPSCs were created first and the BRN3b-meGFP and VSX2-Cerulean components were simultaneously delivered to verified RCVRN-mCherry hiPSCs. Nucleofected hiPSCs were treated with puromycin to select for the VSX2-Cerulean modification and blasticidin to select for the BRN3B-meGFP modification. The PCR strategy used to identify appropriately targeted clones is described in FIG. 1.

The general inventive concepts provide a powerful tool for real time retinal disease modeling. The retinal organoids derived from the general inventive concepts could also provide a platform to test drugs or chemicals to treat various retina diseases or for retinal toxicity. A number of different physical or chemical insults can result in photoreceptor damage, making the search for compounds that can prevent or minimize such damage of great importance for preserving vision. Retinal organoids made from mouse iPSCs, engineered with an Nrl-eGFP reporter to label rod photoreceptors, facilitated the testing of compounds to protect photoreceptors from 4-hydroxytamoxifen-induced degeneration. The PGP1 hiPSC line could easily be used in a similar way with the added advantage of simultaneously monitoring retinal progenitors, bipolar cells and ganglion cells.

The inherent reporters in the PGP1 line will facilitate optimization of protocols to achieve the differentiation and/or survival of specific retinal cell types.

PGP1-derived retinal organoids would permit similar protocol optimization for multiple cell types in real time without relying on antibody labels. The ability to monitor particular cell types continuously without interruption should improve optimization for the survival of desired cells. Specifically, one of the greatest challenges with current retinal organoid technology is achieving long-term survival of retinal ganglion cells.

The PGP1 line could provide an effective strategy for improved purification of specific cell types, cither from retinal organoids or from protocols designed to achieve direct retinal cell type differentiation. During the creation of hiPSC-derived retinal organoids and during normal retinal development, retinal cell types develop in concert. Even in direct differentiation protocols to achieve a particular retinal cell type from hiPSCs, purifying fully differentiated, mature cell types from partially differentiated progenitors remains an issue. The purification of specific retinal neuron cell types can often facilitate downstream analysis, drug screening, or isolation of particular cell populations for transplantation studies. One unique feature of the PGP1 line versus previously reported lines lies in its potential for simultaneously purifying retinal progenitor cells, retinal ganglion cells, photoreceptors and bipolar cells.

PGP1-derived cells will make it possible to evaluate the fate of transplanted cells in vivo. In these transplants, PGP1 progenitors, and later bipolar cells, would exhibit blue fluorescence, while ganglion cells or photoreceptors derived from the transplant would exhibit green or red fluorescence, respectively. In another study, transplantation of GFP-labeled human photoreceptors into a rat model of retinitis pigmentosa demonstrated restoration of the host rod function³⁰. Although these authors reported integration of GFP positive photoreceptors into the outer nuclear layer, the recent realization of cytoplasmic transfer from donor to host cells in photoreceptor transplantation necessitates further confirmation of donor cell integration. mCherry positive photoreceptors from the PGP1 line could provide utility to not only repeat these experiments, but to also serve as an indicator of both cell survival and cell fate of the transplanted photoreceptors.

In certain embodiments, the general inventive concepts are directed to a transgenic cell line (PGP1) that has been modified to express the blue fluorescent protein Cerulean under the control of the endogenous human VSX2 promoter that is specifically active in proliferating neural retina progenitor cells and later becomes restricted to post-mitotic retinal bipolar cells. The PGP1 line also contains a cell membrane localized enhanced green fluorescent protein (eGFP) under the control of the endogenous human BRN3b promoter that is specifically expressed in neural retinal ganglion cells. The third fluorescent reporter in the PGP1 cell line is a red fluorescent mCherry coding sequence under the control of the endogenous human RCVRN promoter, specifically active in retinal photoreceptors (rods and cones).

In summary, Applicants demonstrate a CRISPR/Cas9 strategy to target the expression of multiple fluorescent reporter genes into endogenous loci in hiPSCs. In doing so, Applicants created a triple transgenic hiPSC line (PGP1) and tested the function of this line by directed differentiation into 3D retinal organoids. Organoids produced from the PGP1 line expressed Cerulean in neural retina progenitors and bipolar cells, membrane-targeted eGFP in retinal ganglion cells and mCherry in photoreceptors. In addition, PGP1-derived retinal organoids contained all major retinal cell types. The usefulness of this strategy extends to virtually any cell-type-specific gene, limited only by the number of different fluorescent reporters available for simultaneous analysis.

The PGP1 line, and subsequent hiPSC lines developed using this approach, hold great promise for studying retinal development, disease modeling, drug screening, and pre-clinical transplantation studies.

Accordingly, in an exemplary embodiment, the general inventive concepts contemplate a method for the production of retinal tissue, including retina organoids. The method comprises providing hiPSCs, editing at least one neural retina-specific gene. In certain embodiments, the method comprises editing the stop codon of the at least one neural retina-specific gene. In certain embodiments, the method comprises determining the development of the tissue by monitoring an output resulting from expression of the at least one gene. In certain embodiments, determining comprises applying a particular wavelength of light. In certain embodiments, the retina-specific gene is a gene associated with a particular stage of retina development. In certain embodiments, the retina-specific gene is selected from VSX2 (e.g., SEQ ID NO: 1, SEQ ID NO: 13), BRN3b (e.g., SEQ ID NO: 20, SEQ ID NO: 29), and RCVRN (e.g., SEQ ID NO: 38, SEQ ID NO: 47). In certain embodiments, the method comprises determining the development of the tissue by monitoring an output resulting from expression of the at least one gene. In certain embodiments, the method comprises inserting a fluorescent reporter gene in the retina-specific gene. In certain embodiments, editing comprises inserting a fluorescent reporter gene into more than one retina-specific gene. In certain embodiments, the method comprises inserting a different fluorescent reporter gene into each of the at least one retina-specific genes, including inserting a unique fluorescent reporter gene into each of three retina-specific genes. In certain embodiments, the fluorescent reporter gene is stably expressed.

In certain exemplary embodiments, the general inventive concepts are directed to genetic modification to replace the stop codon of certain endogenous genes with a P2A-fluorescent protein fusion in such a way so as to not destroy the function of the targeted allele. The P2A peptide permits cleavage of the endogenous protein from the fluorescent reporter protein during the process of protein translation. The expression of each reporter gene may be confirmed by differentiating human retinal organoids from the line. Importantly, these reporters express sequentially during retinal development. VSX2 (Cerulean) comes on first, followed by BRN3b (eGFP) with mCherry (RCVRN) coming on last. This makes the PGP1 cell line uniquely capable of following retinal development through time and could provide a readout on drug toxicity for specific retinal cell types.

In certain exemplary embodiments, the general inventive concepts are directed to an expression vector comprising a first nucleic acid molecule encoding a fluorescent protein. In certain exemplary embodiments, the general inventive concepts contemplate paring the first nucleic acid molecule with a second nucleic acid molecule encoding a fluorescent protein. In certain exemplary embodiments, the general inventive concepts contemplate the combination of a first nucleic acid molecule encoding a first fluorescent protein, a second nucleic acid molecule encoding a second fluorescent protein, and a third nucleic acid molecule encoding a third fluorescent protein. In certain exemplary embodiments, the first nucleic acid molecule, second nucleic acid molecule, and third nucleic acid molecule are on the same expression vector. In certain exemplary embodiments, the nucleic acid molecules are on separate expression vectors.

In an exemplary embodiment, the general inventive concepts contemplate a vector for editing at least one neural retina-specific gene. In certain embodiments, the vector is directed to label a particular neural retina cell type. In certain embodiments, the vector is targeted at a retina-specific gene. In certain embodiments, the retina-specific gene is a gene associated with a particular stage of retina development. In certain embodiments, retina-specific gene is selected from VSX2, BRN3b, and RCVRN. In certain embodiments, the vector comprises a fluorescent reporter gene. In certain embodiments, the fluorescent reporter gene is inserted into the stop codon of retina-specific gene.

In certain exemplary embodiments, the general inventive concepts are directed to a transfection kit comprising an expression vector comprising a first nucleic acid molecule encoding a first fluorescent protein. In certain exemplary embodiments, the transfection kit comprises at least one expression vector comprising at least one nucleic acid molecule encoding at least one fluorescent protein. In certain exemplary embodiments, the transfection kit comprises an expression vector comprising a first nucleic acid molecule encoding a first fluorescent protein, a second nucleic acid molecule encoding a second fluorescent protein, and a third nucleic acid molecule encoding a third fluorescent protein.

### Examples

The following examples describe various compositions and methods for genetic modification of cells to aid in the development of retinal organoids, according to the general inventive concepts.

CRISPR/Cas9-based genome editing was used to replace the stop codons of three different neural retina-specific genes with an in frame P2A peptide followed by a fluorescent protein coding sequence in human induced pluripotent stem cells (hiPSCs). Specifically, the stop codon of VSX2 was replaced with a P2A-Cerulean cassette, the stop codon of BRN3B was replaced with a P2A-meGFP cassette, and the stop codon of RCVRN was replaced with a P2A-mCherry cassette, The modification of each of these three genes makes it possible to follow the fate of cells derived from these hiPSCs. When given the appropriate wavelength of light, neural retina progenitor cells derived from these hiPSCs will emit blue fluorescence, retinal ganglion cells and photoreceptor cells will emit green and red fluorescence, respectively. In particular, the membrane-targeted enhanced green fluorescent protein (meGFP) targeted to retinal ganglion cells will make it possible to follow retinal ganglion cell axons from the retina to the brain. As the retina becomes mature, VSX2-Cerulean expression will specifically mark retinal bipolar cells. The inclusion of the P2A peptide should avoid disrupting the function of the targeted alleles. This is because the P2A peptide will facilitate cleavage of the target gene protein from the fluorescent protein during the process of translation. In each case, the hiPSC line is heterozygous for the targeted gene.

FIG. 1 shows CRISPR/Cas9 based strategy to replace the stop codon of the targeted locus with a P2A/Fluorescent reporter fusion gene by homology direct repair.

For transfection, Applicants followed the Lonza Amaxa 4D-Nucleofector Basic Protocol for Human Stem Cells. hiPSCs were nucleofected with two vectors: (1) a sgRNA/Cas9 nuclease, and (2) a dsDNA repair template
sgRNA: a specific gRNA direct Cas9 protein to the appropriate location for cleavage.
   The specific RNA was chosen based on the selection results from three different software tools: (1) https://CRISPR.MIT.edu; (2) https://benchling.com/crispr; (3) http://www.crisprscan.org
dsDNA repair template:
   The repair template will contain ˜700 bp of homology on both sides of the target site, along with the 2A peptide, fluorescent reporter gene and an Frt-flanked with specific antibiotic resistance cassette for positive selection and Thymidine Kinases for negative selection (FIG. 1A).

Following nucleofection, targeted cells were selected with specific antibiotic Antibiotic-resistant hiPSC clones were screened by PCR and sequence analysis using primers specific to the genomic region outside of the homology arm paired with internal primer sequences at each end of the modification (FW1+RV1 and FW2+RV2, blue arrows, FIG. 1B).

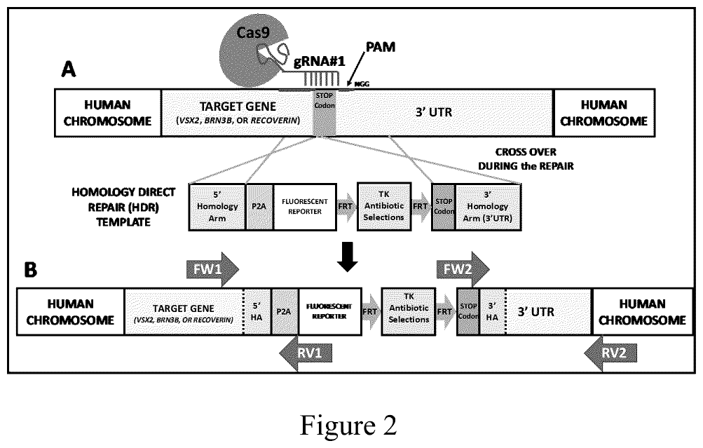

FIG. 2 shows the generation of the hiPSC triple transgenic line.

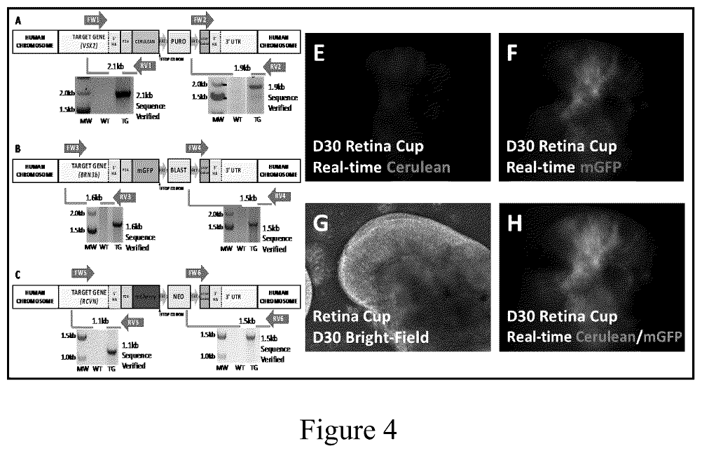

Specifically, to create the hiPSC triple transgenic line, Applicants first created hiPSC/RCVN.mCherry single transgenic line. To make this line:
hiPSCs were nucleofected two vectors: (1) a RCVN-sgRNA/Cas9 nuclease, and (2) RCVN dsDNA repair template.
3 days after nucleofection, hiPSCs were selected with 250 ug/ml of G418 antibiotic for 4 days.
Individual antibiotic-resistant hiPSC clones were isolated, screened by PCR, and sequence analyzed using primers FW5/RV5 and FW6/RV6 (FIG. 2C).
After functionally analyzed the hiPSC/RCVN.mCherry transgenic line using 3-D retinal cups formation, I nucleofected four vectors: (1) a VSX2-sgRNA/Cas9 nuclease, and (2) VSX2 dsDNA repair template, (3) a BRN3b-sgRNA/Cas9 nuclease, and (2) BRN3b dsDNA repair template.
3 days after nucleofection, hiPSCs were selected with double antibiotics: 250 μg/ml of Puromycin and 25 μg/ml of Blasticidin for 4 days.
Two out of three identified triple knock-in clones were used for functional analysis via 3D retina cups formation.
At Day 30 of retina cups differentiation, both of our clones exhibited Cerulean and Green Fluorescence Proteins, which mark for the expressions of VSX2 and BRN3b proteins in real-time (FIG. 4).
The mCherry expected to express around D60 of the retina cups formation, which marks for the RCVN protein real-time.

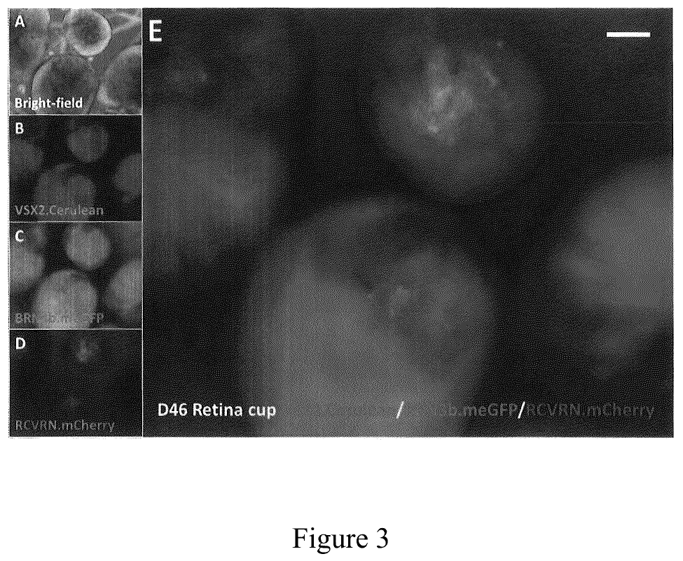

FIG. 3 shows human retinal organoids produced from the triple-targeted hiPSCs, 46 days after the initiation of organoid culture. (A) phase contrast image of hiPSC-derived retina organiods. (B) hiPSC-derived retinal organoids viewed under fluorescence for the excitation of Cerulean to reveal retina progenitor cells. (C) hiPSC-derived retinal organoids viewed under fluorescence for the excitation of cell membrane-targeted eGFP to reveal retinal ganglion cells. (D) hiPSC-derived retinal organoids viewed under fluorescence for the excitation of mCherry to reveal retinal photoreceptor neurons (rods and cones). E. Composite of the merged images B-D.

The following Examples discuss the Creation of the Neural Retina Progenitor/Retinal Ganglion/Photoreceptor (PGP1) Reporter hiPSC Line by CRISPR/Cas9 Genome Editing in greater detail.

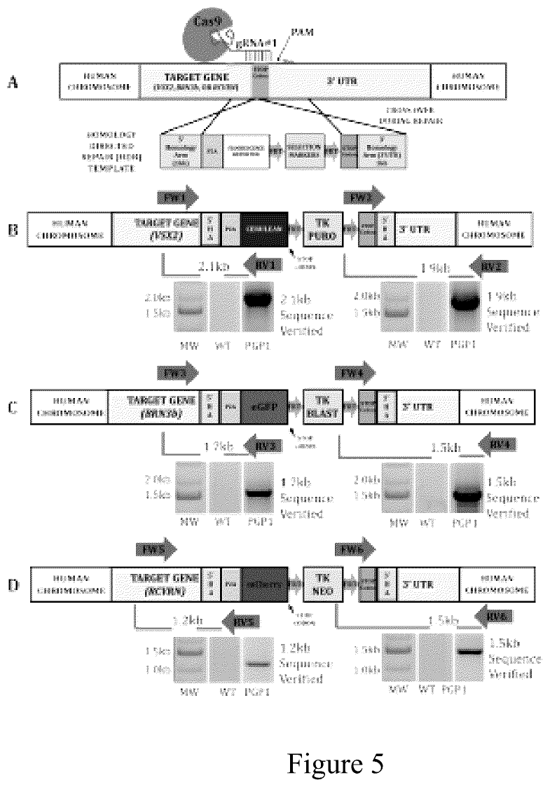

As previously discussed, in order to achieve cell-type-specific fluorescent protein expression without inactivating the targeted gene, Applicants designed a strategy to replace the stop codon of endogenous genes with a sequence encoding a P2A peptide fused to a fluorescent reporter gene by homology directed repair (FIG. 5 A). To facilitate the insertion of multiple different reporter genes, the HDR targeting construct for each gene contained a different antibiotic resistance cassette. Cells nucleofected with both a CRISPR/Cas9 vector (containing the S. pyogenes Cas9 coding sequence and a sgRNA), and an HDR targeting construct were selected with the appropriate antibiotics. DNA sequence analysis of at least two PCR amplicons (Table 1 for primer sequences) encompassing the 5′ and 3′ ends of the targeted modification with at least one PCR primer outside of the sequence contained in the homology arm (HA) confirmed each homologous recombination event (FIG. 5 B-D).

TABLE 1

| Location | Forward (5′-3′) | Reverse (5′-3′) | Size |
|---|---|---|---|
| Outside VSX2 5′HA | CCAAGTGGAGGAAGCGGGAGAAGT | CGGCGGCGGTCACGAAC | 2053 bp |
| to Cerulean | (FW1) | (RV1) | |
| Puro to outside | GCGTTGGCTACCCGTGAT | GCCCCAGCTCCTTATTCC | 1870 bp |
| VSX2 3′HA | (FW2) | (RV2) | |
| Outside BRN3b 5′HA | TATTCGGCGGGCTGGATGAGAGTC | GCCGTCGCCGATGGGGGTGTT | 1673 bp |
| to eGFP | (FW3) | (RV3) | |
| Bias to outside | TCGACTAGAGCTTGCGGAACC | AACCAGGCCATATACAGAACTCAA | 1528 bp |
| BRN3b 3′HA | (FW4) | (RV4) | |
| Outside RCVRN 5′HA | AGCTTTGTTGAGCACCGACT | GTTCTCCTCCACGTCTCCAG | 1167 bp |
| to mCherry | (FW5) | (RV5) | |
| Neo to outside | TCGCCTTCTTGACGAGTTCT | TGGATCTGGTCCTCTCCATC | 1493 bp |
| RCVRN 3′HA | (FW6) | (RV6) | |
| Outside VSX2 5′HA | CCAAGTGGAGGAAGCGGGAGAAGT | GCCCCAGCTCCTTATTCC | 2627 bp |
| to outside VSX2 3′HA | (FW1) | (RV2) | |
| Outside BRN3b 5′HA | TATTCGGCGGGCTGGATGAGAGTC | AACCAGGCCATATACAGAACTCAA | 2438 bp |
| to outside BRN3b 3′HA | (FW3) | (RV4) | |
| Outside RCVRN 5′HA | AGCTTTGTTGAGCACCGACT | TGGATCTGGTCCTCTCCATC | 2221 bp |
| to outside RCVRN 3′HA | (FW5) | (RV6) | |

The creation of the PGP1 line involved two sequential rounds of CRISPR/Cas9 genome editing. The first step consisted of targeting the RCVRN locus in wild-type (WT) hiPSCs with the mCherry fluorescent protein followed by selection with G418 (FIG. 5 D). Forty-eight of the G418 resistant hiPSC clones tested by PCR analysis (blue arrows) and DNA sequencing identified three (6.25%) correctly targeted clones. Simultaneous targeting of a G418 resistant RCVRN-targeted hiPSC clone with sgRNAs and HDR targeting constructs for VSX2 and BRN3b (FIG. 5 B, C) resulted in the selection (puromycin (puromycin resistant—SEQ ID NO: 9) and blasticidin (blasticidin resistant—SEQ ID NO: 27) of 144 resistant clones. Of these triple resistant clones, four (2.8%) were correctly targeted at the VSX2, BRN3b, and RCVRN, loci. Of the three triple targeted hiPSC lines isolated, Applicants conducted our subsequent analysis on one line that Applicants designated PGP1.

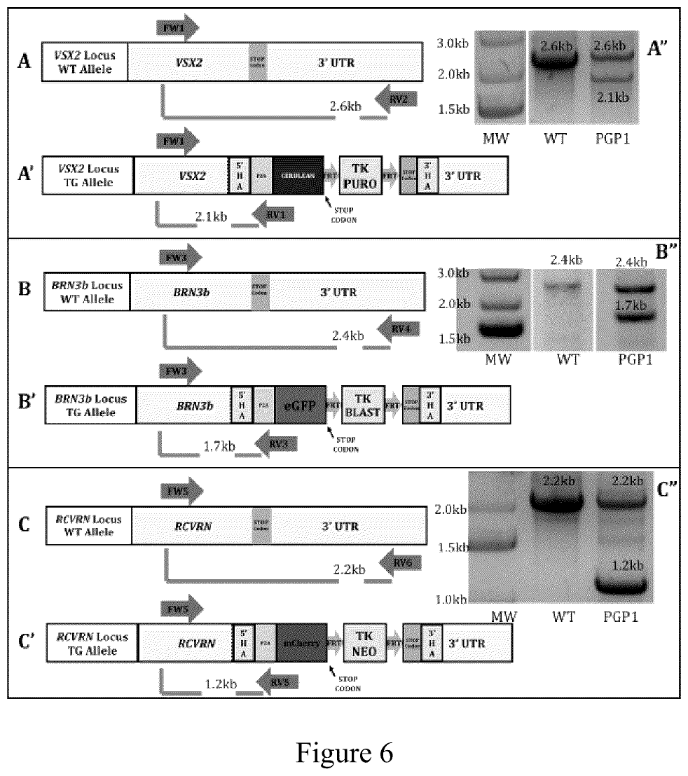

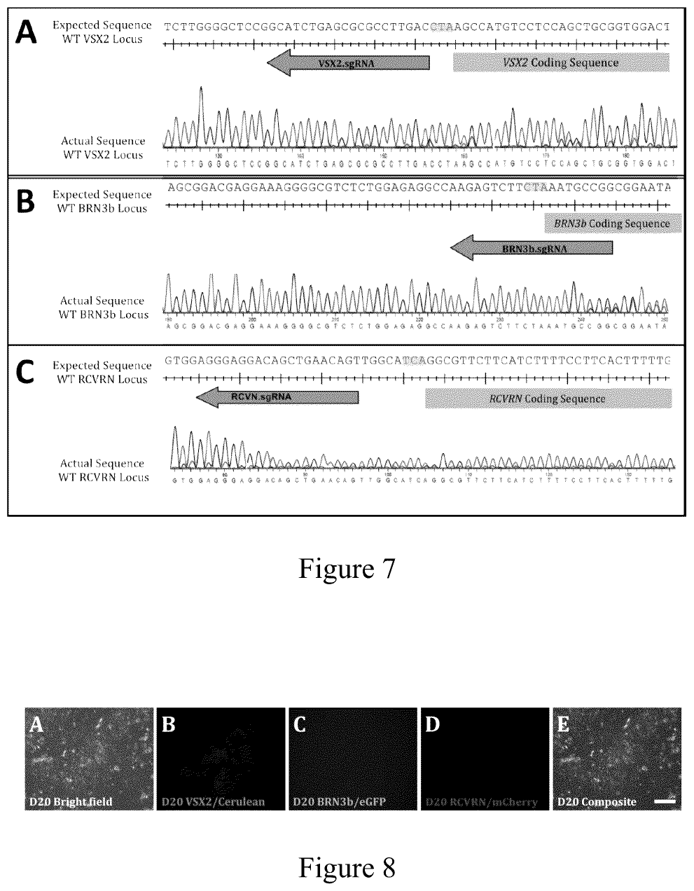

To characterize the molecular features of the PGP1 line, Applicants used primers for PCR and DNA sequencing to analyze both alleles of the targeted genes as well as to screen for potential Cas9-generated off-target mutations. A three-primer PCR strategy, utilizing primers introduced in FIG. 5, simultaneously tested for both the targeted and WT alleles. The forward primer used for screening the 5′ targeting event acted as a common primer from the endogenous gene. Two different reverse primers, one made to the fluorescent reporter gene specific for the targeted allele, and one made to the 3′ untranslated region of the endogenous gene specific for the WT allele, made it possible to determine if the clone was heterozygous or homozygous for the targeting event. PCR amplification produced DNA fragments of the expected size for both WT and targeted alleles for each gene from PGP1 genomic DNA (FIG. 6). Sequence analysis of the DNA band consistent with the WT allele from each target gene size failed to reveal any indel mutations at the sgRNA cut site (FIG. 7). DNA sequencing facilitated the analysis of PCR fragments amplified by primers surrounding predicted high-scoring off-target sites for each sgRNA. This analysis revealed no off-target mutations in PGP1 (FIG. 7 and Tables 2-4).

TABLE 2
Off-Target Screening VSX2-sgRNA

| Name | Gene | Sequence | PAM | Off-target Score* |
|---|---|---|---|---|
| VSX2, Chr14 | | GTCAAGGCGCGCTCAGATGC | CGG | 100 |
| Chr19 non-gene | | GTCAAGGCGTACTCAGATGC | GAG | 2.668116758 |
| sequence | | | | |
| Chr19 non-gene | | GTGAAGAAGTGCTCAGATGC | CAG | 0.916843223 |
| sequence | | | | |
| Sytabulin, Chr8 | ENSG00000147642 | GTGAAGACACCCTCAGATGC | TGG | 0.349165048 |
| VEGF-A, Chr6 | ENSG00000112715 | GTCAAGGCGTGCTCCGATGG | GGG | 0.317986706 |
| KIF16B, Chr20 | ENSG00000089177 | GTCGAAGCGGGCTCCGATGC | AGG | 0.252128661 |
| STAT2, Chr12 | ENSG00000170581 | GTCAATGGGAGCTCTGATGC | AGG | 0.234357477 |

TABLE 3
Off-Target Screening BRN3b-sgRNA

| Name | Gene | Sequence | PAM | Off-target Score* |
|---|---|---|---|---|
| BRN3b (POU4F2), | ENSG00000151615 | AAGAGTCTTCTAAATGCCGG | CGG | 100 |
| Chr4 | | | | |
| RP11-1100L3.7, | ENSG00000257663 | AGCAGTCTTCCAGATGCCGG | CAG | 0.371654759 |
| Chr12 | | | | |
| RP11-45M11.7, | ENSG00000275846 | AAGCTCCTTCTAAATGCCAG | TAG | 0.351773802 |
| Ch6 | | | | |
| TC2N, Chr14 | ENSG00000165929 | TAAAGTCTTCTAAATGCCAA | TAG | 0.331943062 |
| FAM83F, Chr22 | ENSG00000133477 | AAGAGAATTGGAAATGCCGG | CAG | 0.302192873 |
| RP11-484K9.4, | ENSG00000272844 | AAGACTCTTTGAAATGCCTG | CGG | 0.288247111 |
| Chr3 | | | | |
| RP11-321M21.1, | ENSG00000266774 | AATAGTCCTCCAAATGCTGG | CAG | 0.202171083 |
| Chr18 | | | | |

TABLE 4
Off-Target Screening RCVRN-sgRNA

| | | | | Off- |
|---|---|---|---|---|
| Name | Gene | Sequence | PAM | target Score* |
| Recoverin, | ENSG00000109047 | AGGGAGGACAGCTGAACAGT | TGG | 100 |
| Chr17 | | | | |
| Chr4 non-gene | | AGGGAGGCCAGCTGAAGAGT | GGG | 3.099576271 |
| sequence | | | | |
| Chr2 non-gene | | GGAGAGGGCAGCTGAACAGT | TAG | 2.726928675 |
| sequence | | | | |
| Chr14 non-gene | | AGAGAGATCAGCTGAACAGT | GGG | 1.740860136 |
| sequence | | | | |
| Chrl7 non-gene | | AGAAAGGACAGCTGAACTGT | AGG | 0.741790707 |
| sequence | | | | |
| Chr17 non-gene | | AGTGAGGATAGCTGGACAGT | AGG | 0.541032634 |
| sequence | | | | |

### Functional Analysis of the Fluorescent Reporters in the PGP1 Line

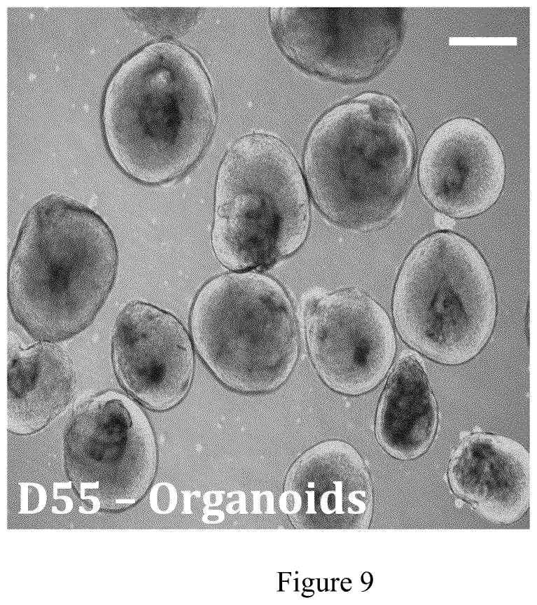

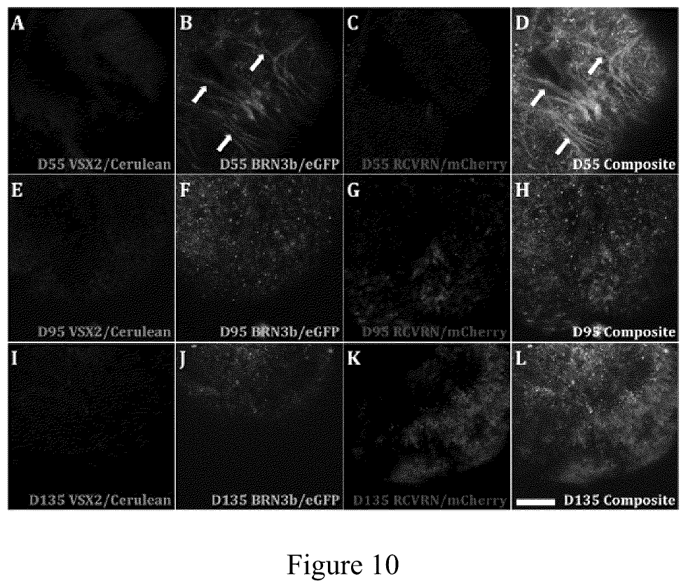

To confirm that all three fluorescent reporter genes would express appropriately during retinal differentiation, retinal organoids from the PGP1 hiPSCs were created. Retinal organoids were differentiated from PGP1 hiPSC-derived embryoid bodies as previously described. Initially, free-floating embryoids were seeded onto Matrigel-coated plates to allow for the formation of eye field domains. At day 20 (D20) of differentiation, these eye field domains expressed Cerulean but not eGFP or mCherry (FIG. 8). After four weeks of differentiation, Cerulean positive retinal domains were manually detached from the Matrigel-coated plates and cultured as free-floating 3-dimensional retinal organoids. These organoids formed spherical cups after 55 days of differentiation (FIG. 9). By day 55 (D55) organoids revealed widespread fluorescence of Cerulean protein, expressed from the VSX2 promoter (FIG. 10 A). Likewise, at D55 these organoids exhibited eGFP expression in a more limited population of cells, driven by the BRN3b promoter, and these eGFP-expressing cells preferentially localized to the interior portion of the organoids (FIG. 10 B). During construction of the BRN3b/eGFP targeting vector, Applicants inserted a cell membrane signal peptide tag (GAP43 palmitoylation sequence) to drive membrane eGFP fluorescence¹⁷. This modification facilitated visualization of developing retinal ganglion cell axons at this stage. At D55, mCherry fluorescence, driven by the RCVRN promoter, appeared sporadically in a few cells throughout the organoids (FIG. 10 C). A merged image of all three fluorescent signals shows the restriction of fluorescent protein expression to distinct cells with little evidence of overlapping expression of different reporters (FIG. 10 D).

As the organoids continued to mature, layering of the fluorescent cell types became more distinct and the ratio of cells expressing different fluorescent proteins changed. At D95, Cerulean expression largely occupied an intermediate layer within the organoid (FIG. 9E, H) with eGFP concentrated on the organoid interior (FIG. 10 F, H) and mCherry expressing cells organized along the organoid periphery (FIG. 10 G, H). Although the number of eGFP-expressing cells increased from D55 to D95, elongated ganglion cell axons were not as distinguishable as they were at D55. By D135, the organoids had increased in size with Cerulean and eGFP positive cells occupying space in the interior of the organoid with abundant mCherry fluorescence apparent in the peripheral layers of the organoid (FIG. 10 I-L).

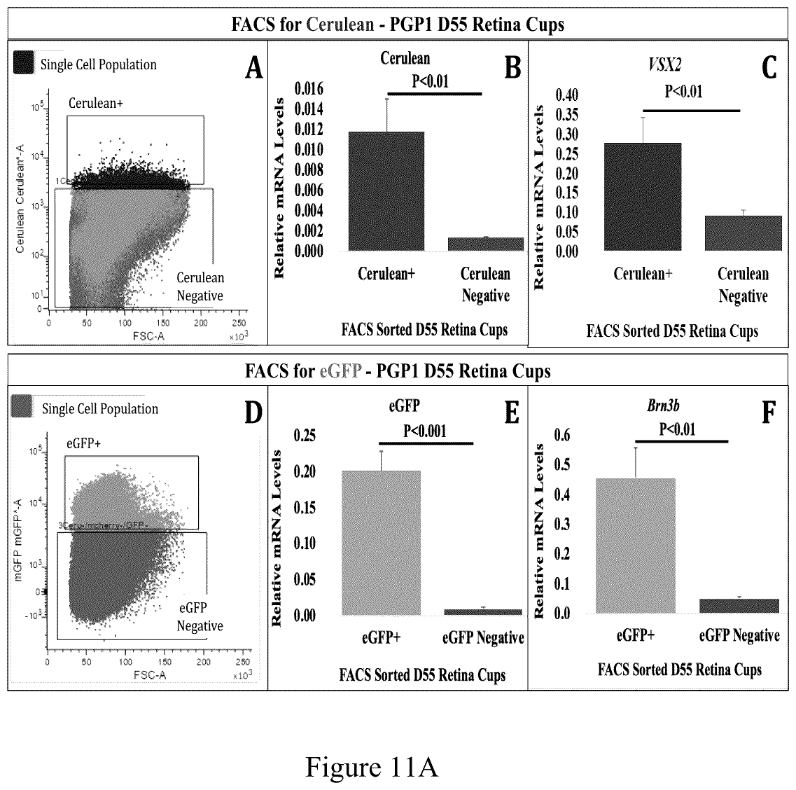

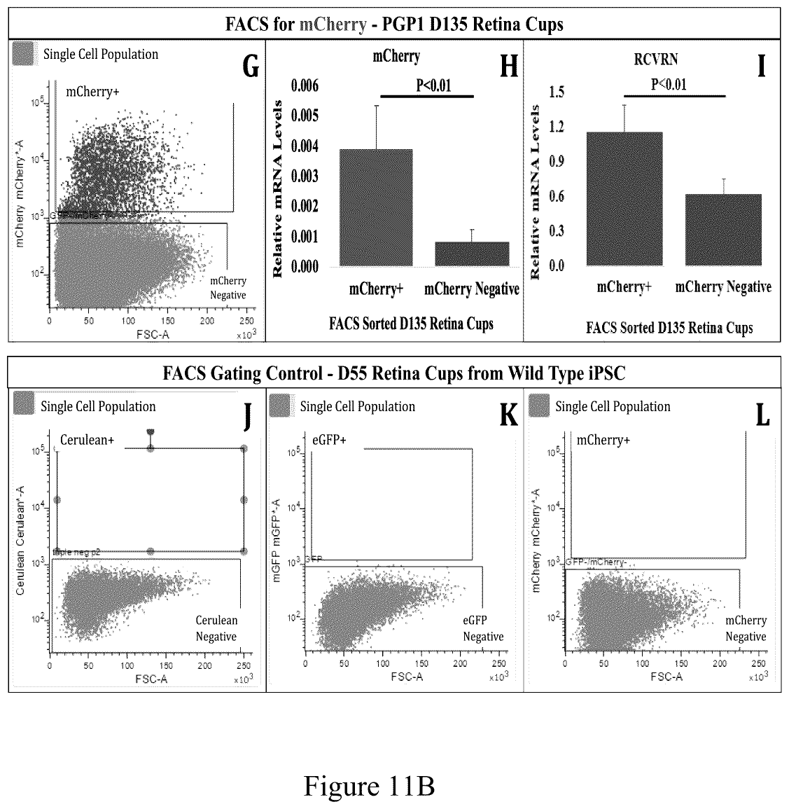

To confirm that the observed fluorescent protein expression in PGP1-derived retinal organoids corresponded to the appropriately targeted cell types, Applicants sorted cells from the organoids based on fluorescent protein expression. At D55, the organoids contained many cells positive for Cerulean and eGFP, but relatively few mCherry positive cells. However, by D135 the organoids contained abundant mCherry positive cells. Considering this, Fluorescence Activated Cell Sorting (FACS) was performed on proteolytically disaggregated single cell suspensions to collect Cerulean (FIG. 11A) and eGFP (FIG. 11 D) expressing cells from D55 organoids and collected mCherry (FIG. 11 G) expressing cells from D135 organoids. Gates for collection of appropriate fluorescent cells were established by sorting human embryonic kidney (HEK293) cells transiently transfected with plasmids directing constitutive expression of Cerulean, eGFP or mCherry. At D55, Cerulean positive cells made up approximately 11.3%, and eGFP positive cells comprised approximately 4.9% of the single cells from disaggregated organoids consisting of 728,023 total cells. At D135, approximately 53% of the organoid cells (1,080,000 total cells) were mCherry positive. Applicants also sorted organoids from wild type hiPSCs using the same gates used for the PGP1-derived organoids and found no cells within the Cerulean (FIG. 11 J), eGFP (FIG. 11 K) or mCherry (FIG. 11 L) gates.

To validate our FACS gating and explore the nature of the sorted populations, Applicants performed reverse transcription-quantitative PCR (RT-qPCR) analysis on sorted populations of cells. In each case, Applicants sorted disaggregated organoids independently for each color into three separate tubes and compared cells positive for each sorted color to the population of cells that were negative for that sorted color according to our pre-established gates. Each of the three sorts was tested by RT-qPCR in triplicate, meaning that all graphical data represented nine RT-qPCR reactions. As expected, the Cerulean positive population expressed significantly more Cerulean mRNA than the Cerulean negative population (FIG. 11B). Likewise, the Cerulean positive population expressed approximately 3 times more VSX2 mRNA than the Cerulean negative population (FIG. 11 C). The eGFP positive sorted population expressed 22 fold more eGFP mRNA and 9.1 fold more BRN3b mRNA than the eGFP negative population of cells (FIG. 11 E, F). On D135, the mCherry positive sorted population expressed approximately 4.9 times more mCherry mRNA and 1.9 times more RCVRN mRNA than the mCherry negative sorted population (FIG. 11 H, I). This demonstrates that the fluorescent reporters faithfully reveal the cells expressing the gene to which they were targeted. For both VSX2 and BRN3b the large majority of expressing cells appeared in the Cerulean and eGFP positive populations, respectively. The appearance of relatively more RCVRN transcripts in the mCherry-negative population (although significantly less than RCVRN expression in the mCherry positive population) suggests that not all mCherry-expressing cells were captured within our gate. Alternatively, it is possible that the targeted RCVRN allele expresses RCVRN and mCherry less efficiently than the non-targeted RCVRN allele.

### PGP1-Derived Retina Organoids Contain All Major Retina Cell Types

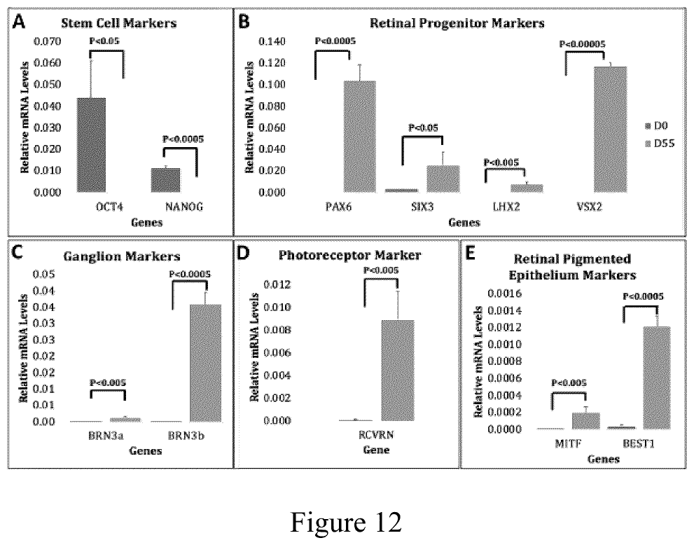

A comparison of transcripts expressed by PGP1 hiPSCs at D0 of differentiation with those expressed by PGP1-derived retinal organoids at D55 revealed a marked decrease in pluripotency-related genes and a significant increase in retina differentiation-related genes. Pluripotency transcripts for OCT4 (POU5F1) and NANOG expressed abundantly in PGP1-hiPSCs at D0 but virtually disappeared in the D55 organoids (FIG. 12 A). In contrast, the retinal progenitor transcripts for PAX6, SIX3, LHX2 and VSX2 increased significantly during early retinal organoid differentiation (FIG. 12 B). Transcripts for ganglion cells (BRN3a and BRN3b), photoreceptors (RCVRN) and retina pigment epithelium (MITF and BEST1) also increased significantly from D0 to D55 of differentiation (FIG. 12 C, D, E).

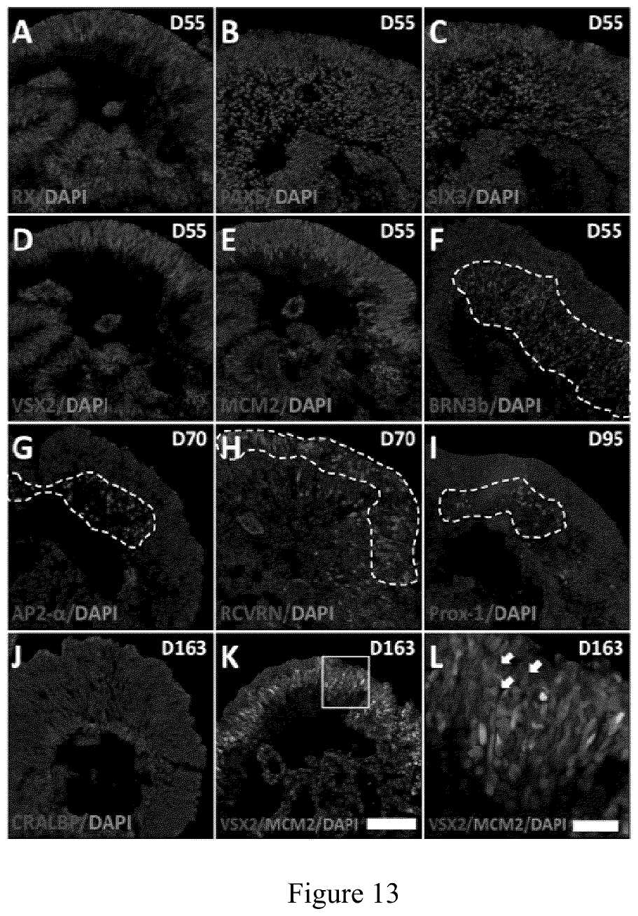

PGP1-derived retinal organoids contained cells matching the immunological profile of all major retinal cell types. At D55, the organoids expressed the eye field precursor RX (FIG. 13 A), retinal progenitor markers: PAX6, SIX3, VSX2 (FIG. 12 B, C, D), and the proliferation marker MCM2 (FIG. 13 E). All of these aforementioned proteins appeared throughout the D55 organoid tissue. In contrast, the expression of BRN3b, a protein characteristic of retinal ganglion cells, appeared largely absent from the outer layer of the organoid, occupying a distinct localization within the organoid interior (outlined in FIG. 13 F). By D70, AP-2α, a protein expressed by amacrine cells, also appeared in the interior region of the organoid (outlined in FIG. 13 G). Although RCVRN expression, marking differentiating rods and cones, initially appeared throughout the organoid (not shown), by D70 RCVRN expressing cells appeared most prominently near the outer edge of the organoids (outlined in FIG. 13 H). At D95, PROX1 expression, characteristic of horizontal cells, occupied a space below the putative outer nuclear layer of the retina organoid (outlined in FIG. 13 I). In retinal organoids, Müller glia cells represent a late differentiating cell type. Applicants observed CRALBP expression, characteristic of Müller glial cells, through the full thickness of the retinal organoids at D163 (FIG. 13 J). Although VSX2 expression characterizes proliferating (MCM2 positive) retina progenitor cells, VSX2 re-appears in differentiated, non-proliferating (MCM2 negative) bipolar cells by D95 (not shown). These VSX2 positive/MCM2 negative bipolar cells increase in abundance by D163 (FIG. 13 K, L).

### Methods

hiPSC Culture: Human umbilical cord stem cell derived hiPSC6.2 (Life Technologies, A18945) were grown on Matrigel-coated (MG) plates using chemically defined Essential 8 medium (Thermo fisher, A1517001) as described previously. The medium was changed daily, and cells were passaged every 3-4 days using 0.5 mM EDTA in 1×DPBS without calcium and magnesium to lift cells from the tissue culture plate (Thermo fisher, 15575020).

Specific Single Guide RNA Vector (sgRNA) Design: CRISPR specific guide RNAs (sgRNAs) were individually cloned into a U6-driven (U6 promoter—SEQ ID NO: 57) sgRNA expression PX458 (SEQ ID NO: 58, 60, 61, 62, 63, 64, 66, 67, 68, 69, 70, 71, 72, 73) vector that includes the S. pyogenes Cas9 coding sequence (Addgene, #48138, SEQ ID NO: 65) as described. To determine the Cas9 cutting efficiency of these sgRNAs, Applicants transfected each vector into Human Embryonic Kidney (HEK293) cells. Forty-eight hours after transfection, HEK293 DNA was extracted and amplified by Q5 High-Fidelity PCR (NEB, M0494S) using primers encompassing the sgRNA recognition site. The PCR products were digested with T7 Endonuclease I (T7E1, NEB M0302S) according to the recommended protocol. Successful Cas9 cleavage by sgRNAs resulted in two distinct bands in the T7E1 assay.

TABLE 5

| Specific guide RNA | 5′-3′ |
|---|---|
| VSX2.sgRNA | GTCAAGGCGCGCTCAGATGC |
| | (SEQ ID NO: 59) |
| BRN3b.sgRNA | AAGAGTCTTCTAAATGCCGG |
| | (SEQ ID NO: 74) |
| RCVRN.sgRNA | AGGGAGGACAGCTGAACAGT |
| | (SEQ ID NO: 75) |

Homology Directed Repair (HDR) Template Generation: To generate the HDR templates, the left and right homology arms (HA) of each locus were amplified from the WT hiPSC genomic DNA using primers listed in Table 6. The amplified homology arms were inserted into the P2A: Cerulean.pL451 (pL451—SEQ ID NOs: 14, 15, 16, 17, 18, 19, 31, 32, 33, 34, 35, 36, 37, 45, 46, 47, 48, 49, 50, 51, 52, 53, 54, 55, 56); P2A:GAP43.eGFP.pL451, P2A:mCherry.pL451 vectors via Gibson Assembly (NEB37). The Cerulean tag came from addgene #53749, the membrane bound form of eGFP from addgene #14757, and mCherry from addgene #26901. DNA sequencing verified the generated HDR templates following PCR amplification.

TABLE 6

| Gene | Forward (5′-3′) | Reverse (5′-3′) |
|---|---|---|
| VSX2.5′HA | ATTGGGTACCGGGCCTC | CCGCTTCCGTCGACCAA |
| | CTGTGAGAACAGTGTG | AGCCATGTCCTCCAGC |
| VSX2.3′HA | ATACGAAGTTATTAGGT | CTCCACCGCGGTGGCGC |
| | GTAGGTCAAGGCGCGCT | CAGATTGGGTTGTTCAA |
| | CA | GG |
| RCVRN.5′HA | CTATAGGGCGAATTGGG | GTCGACCTCGAGGGGGG |
| | TACTGCCTTCCCCGCCA | GCCTGGCGTTCTTCATC |
| | GGTC | TTTTCCTTCACTTTTTG |
| RCVRN.3′HA | ATACGAAGTTATTAGGT | CTCCACCGCGGTGGCCA |
| | GTGAACACACATGCACA | AAAGCTTATTCATCGGG |
| | CA | |
| BRN3b.5′HA | GGCGAATTGGAGCTCCA | ATACAGCACAGCATAGG |
| | CCGCGGTGGCCGCCGAG | TCCAGGGTTCTCCTCCA |
| | GCTCTGGCAGC | CG |
| BRN3b.3′HA | CCACTAGTTCTAGAAAT | TTGATATCGAATTCCTG |
| | AGAAGACTCTTGGCCTC | CAGCCCGGGGTGCATCG |
| | TCC | GTCATGCTTCC |

Insertion of Fluorescent Reporter Genes into Selected Loci: To generate the RCVRN/mCherry hiPSC lines Applicants transfected 2.5 μg of the HDR template and 2.5 μg of the sgRNA vector into WT hiPSCs using a 4D-Nucleofector X Unit and the P3 Primary Cell kit (Amaxa, V4XP-3012) based on manufacturer's protocol. After transfection, the cells were cultured with Essential 8 media plus 10 μM ROCK inhibitor (Sigma SCM075) overnight. Subsequently, culture media was changed daily without addition of ROCK inhibitor. Forty-eight hours after transfection, antibiotic selection began with 100 μg/ml and slowly increased to 250 μg/ml of G418 (Corning, 30234CR) over the course of one week to select for Neomycin resistant colonies. Resistant clones were manually picked and cultured individually in a 48 well plate. The DNA of each clone was extracted (Zymo Research, D3025), and screened for reporter integration by PCR (Thermo Fisher, EP1701) using the primers listed above. The clones, which exhibited the expected PCR fragment sizes on each side of the HDR junctions, were validated by DNA sequencing. To generate the triple transgenic hiPSCs, a RCVRN/mCherry hiPSC clone was transfected with 1.25 μg each of: the BRN3b/eGFP HDR template, the sgRNA vector to the BRN3b locus, the VSX2/Cerulean HDR template, and the sgRNA vector to the VSX2 locus using nucleofection as described above. Double antibiotic selection was performed using 100 μg/ml Blasticidin and 100 μg/ml Puromycin, and slowly increased up to 250 μg/ml of each antibiotic over the course of one week to select for Blasticidin and Puromycin resistant colonies. Resistant clones were manually picked and cultured in a 48 well plate (one clone per well). Again, DNA from each clone was extracted and screened by PCR using primers listed above. PCR bands of the expected size were verified by DNA sequencing.

Off-Target Screening: The PGP1 clone was screened by selecting five high scoring off-target sites for each sgRNA used according to online tools provided by Benchling. Each potential sgRNA off target site listed in Tables 2-4 (off-target score provided by Benchling) was screened by High-Fidelity PCR (Q5 NEB, M0491L) with primers listed in Tables 7-9 and PCR products were sequenced using Eurofins Genomics tube DNA sequencing services. Each result was independently repeated five times.

TABLE 7
Set of primers used to screen for off-target cutting efficiency of VSX2.sgRNA

| Gene | Forward (5′-3′) | Reverse (5′-3′) | Size |
|---|---|---|---|
| Sytabulin | GCACCGCATGGCTTCTCACC (*) | GGCCCCATCAAAATAAAACCATC | 1.2 kb |
| VEGFA | TGTGGCGGCCTCCCTTCATCTG (*) | CCCGCTCGCTCGCTCGCTCAC | 887 bp |
| Kinesis | GCCTGGCACCCTTGACATT | AGCAGGCAGAGCATCCCATCC (*) | 913 bp |
| Stat2 | TTGAGGGGCTGGAGAAAGATAAGT (*) | TGGGGAGCAGAGACAAATAGAGAA | 906 bp |
| Chr19 | CACTGCCCACTACCCACTACTAAG (*) | CGGGAGCAATATGGGAAATGGTC | 941 bp |

(*) symbol at the end indicates that this primer is good to use as probe for sequencing

TABLE 8
Set of primers used to screen for off-target of cutting efficiency RCVRN.sgRNA

| Gene | Forward (5′-3′) | Reverse (5′-3′) | Size |
|---|---|---|---|
| chr4 | TGTTCCCGGCCATTTGTA (*) | ATCTTGCCAGCATCCATTATCT | 844 bp |
| chr2 | AAGCCCACTGGAAAGGTATGAACT (*) | AATGGGAAGGGGACTGAACAAA | 833 bp |
| chr14 | AGTTTACGGGAGGGAGGTCAGC (*) | TGGCAGGGAGAAACAGTAGAA | 596 bp |
| chr17 | GGGTGGCGGCAGCTTGATAAA (*) | CCCCGAGGATAGCACTGTTGG | 497 bp |
| chr17 | GAGCCCCCGGAAGCACAAATACAG (*) | GGCAGGCGTCTCCGTTCTCACAC | 648 bp |

(*) symbol at the end indicates that this primer is good to use as probe for sequencing

TABLE 9
Set of primers used to screen for off-target cutting efficiency of BRN3b.sgRNA

| Gene | Forward (5′-3′) | Reverse (5′-3′) | Size |
|---|---|---|---|
| 1100L3.7 | CTTCCCGGCACCAAATCACTCTAC (*) | GCCCCTCCCCTGCTTATCTGG | 1.0 kb |
| 45M11.7 | ACCCCTTTTATTCGTGCTCTATTG (*) | AGTCCCGCGTCCTGCTCTC | 1.0 kb |
| FAM83F | TGGCCTTTTGCTTTTTCACACC | CACCCCCGGCGTCCTTTACCTG (*) | 854 bp |
| RP11-484K9 | CCGTAGGGGGCGAGGAACC (*) | GTGAAGGCGGAAATACAAACAGTC | 691 bp |
| RP11-321M21 | GGGGCAAGCTTCTCCACTATTATC | GTTCCATCCTGCGGCTCTTC (*) | 931 bp |

(*) symbol at the end indicates that this primer is good to use as probe for sequencing

3-D Retinal Organoid Generation from the PGP1 Triple Targeted Line: The PGP1 line was used to create 3-D retinal organoids as described previously using the Zhong et al., 2014 protocol with the following modifications. Briefly, hiPSC were incubated in 0.5 mM EDTA/DPBS (Thermo fisher, 15575020) for 5 minutes at 37° C. Cells were then dissociated into small clumps and cultured in mTeSR1 medium with 10 μM ROCK inhibitor to form aggregates. The aggregates were gradually transitioned into neural induction medium (NIM) for three days (D1-3 of differentiation), then cultured in NIM from D3 to D6. On D7, the aggregates were seeded on Matrigel (hESC-qualified, Corning)-coated dishes in NIM at an approximate density of 20 aggregates per cm² and switched to DMEM/F12 (3:1) supplemented with 2% Gem21 NeuroPlex (without vitamin A, Gemini Bio-Products, 400-161), 1×NEAA, and 1% antibiotic-antimycotic (Thermo, 15240062) on D16. The medium was changed every 3 days. On the fourth week (D28) of differentiation, a cell scraper was used to detach the cells from the dishes and the cells were transferred to petri dishes. The cells were then cultured in suspension at 37° C. in a humidified 5% CO2 incubator in DMEM/F12 (3:1) supplemented with 2% Gem21 NeuroPlex, 1×NEAA, and 1% antibiotic-antimycotic. Within 3-5 days, cells began forming 3-D retinal organoids. The organoids were then mechanically separated from the rest of the cells using sharpened tungsten needles under a dissecting microscope. From that point on, the medium was changed twice a week. To culture the retinal organoids long-term, the medium was supplemented with 10% fetal bovine serum (Gibco), and 2 mM GlutaMax (Invitrogen) beginning on D42.

Reverse Transcription-PCR Analysis of Retinal Organoids: Total RNA isolation of hiPSC colonies or retinal organoids was done using either Quick RNA miniprep plus (Zymo research, R1057) or 96-well plate RNA extraction kit (Illustra RNAspin 96, 25-0500-75) using the manufacturer's protocol. Reverse transcription was performed using the ImProm-II Reverse Transcription System (Promega, A3800) according to the manufacturer's protocol. RT-qPCR was performed with GoTaq® qPCR Master Mix for Dye-Based Detection (Promega, A6001) using a CFX Connect Bio-Rad qPCR System. Forty cycles were run at 95° C. denaturation for 40 s, at 60° C. annealing for 40 s and at 72° C. extension for 60 s, using primers listed in Table 10. The expression levels of individual genes were normalized to GAPDH mRNA levels and analyzed using the detla-delta Ct method (Applied Biosystems) with significant differences revealed by a two tailed Student's t-test. Error bars in each figure represents the standard error (SE) of three individual experiments.

TABLE 10

| Gene | Forward (5′-3′) | Reverse (5′-3′) |
|---|---|---|
| Oct4 | TGTACTCCTCGGTCCCT | TCCAGGTTTTCTTTCCC |
| | TTC | TAGC |
| NANOG | CAGTCTGGACACTGGCT | CTCGCTGATTAGGCTCC |
| | GAA | AAC |
| PAX6 | CGGAGTGAATCAGCTCG | CCGCTTATACTGGGCTA |
| | GTG | TTTTGC |
| SIX3 | CCGGAAGAGTTGTCCAT | CGACTCGTGTTTGTTGA |
| | GTT | TGG |
| VSX2 | TCATGGCGGAGTATGGG | TCCAGCGACTTTTTGTG |
| | CT | CATC |
| BRN3a | GGGCAAGAGCCATCCTT | CTGTTCATCGTGTGGTA |
| | TCAA | CGTG |
| BRN3b | CTCGCTCGAAGCCTACT | GACGCGCACCACGTTTT |
| | TTG | TC |
| RCVRN | CCAGAGCATCTACGCCA | CCGTCGAGGTTGGAATC |
| | AGTT | GAAG |
| MITF | GACATGCGCTGGAACAA | CCGGGGGACACTGAGGA |
| | GGGAACC | AAGGAG |
| BEST-1 | AACTGAGCCTACCACAC | CGGATTCGACCTCCAAG |
| | AACA | CC |

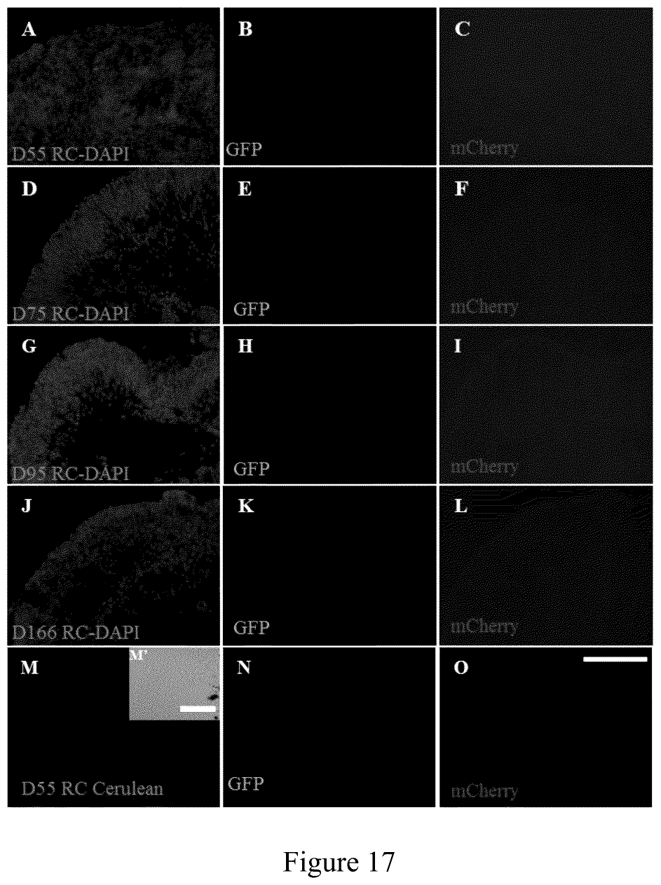

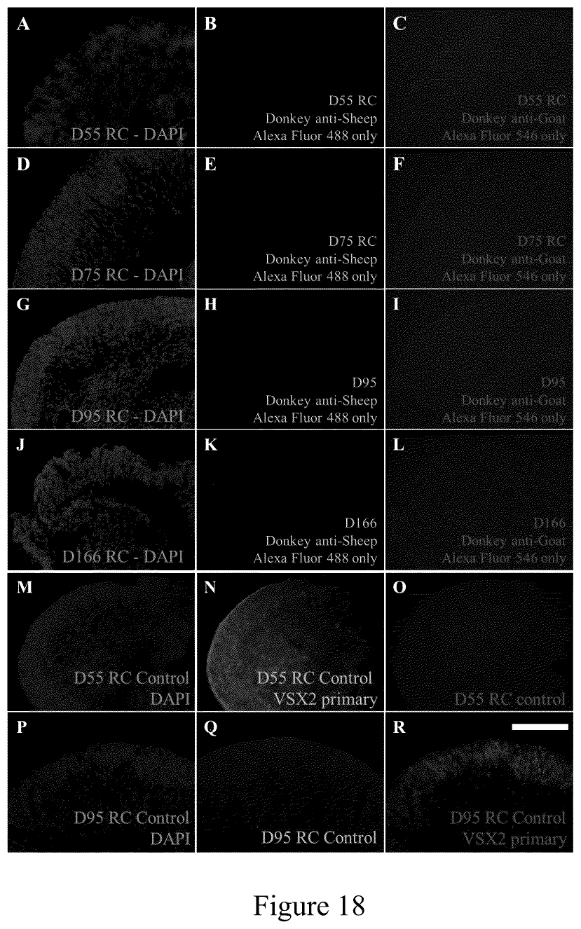

Immunofluorescence of the Retinal Organoids: Retinal organoids were fixed with 4% paraformaldehyde (4% PFA) for 20 minutes at room temperature. The fixed organoids were incubated in 30% sucrose overnight at 4° C. before embedding in OCT. Organoids were cryosectioned at 15 μm prior to immunofluorescence. Immunofluorescence was performed using antibodies specifically against the proteins of interest listed in Table 11. Fluorescent images were acquired with a Nikon Eclipse 80i microscope and/or Zeiss LSM 710 Laser Scanning Confocal System. Fluorescent protein fluorescence does not survive our fixation and embedding protocol and therefore provides no interference with secondary antibody fluorescence (FIGS. 17 and 18).

TABLE 11

| Antibodies | Supplier | Species | Type | Dilution | Reference |
|---|---|---|---|---|---|
| VSX2 | Millipore | Sheep | Polyclonal | 1:500 | ab9016 |
| CFP | Abcam | Rabbit | Polyclonal | 1:100 | ab6556 |
| BRN3 | Santa Cruz | Goat | Polyclonal | 1:1000 | sc-6026X |
| MCM2 | Abcam | Rabbit | Polyclonal | 1:1000 | ab4461 |
| Prox-1 | Millipore | Rabbit | Polyclonal | 1:2000 | ab5475 |
| Cralbp | Abcam | Mouse | Monoclonal | 1:500 | ab15051 |
| Ap2-alpha | DSHB | Mouse | Monoclonal | 1:35 | 3B5a |
| Recoverin | Millipore | Rabbit | Polyclonal | 1:500 | ab5585 |
| Oct4 | Abeam | Rabbit | Polyclonal | 1:500 | ab19857 |
| Sox2 | Santa Cruz | Goat | Polyclonal | 1:500 | Sc-17319 |
| Pax6 | Santa Cruz | Mouse | Polyclonal | 1:100 | Sc-32766 |
| Six3 | Santa Cruz | Mouse | Polyclonal | 1:100 | Sc-365519 |
| Rx | Santa Cruz | Mouse | Polyclonal | 1:150 | Sc-271889 |

TABLE 12

| Gene | Forward (5′-3′) | Reverse (5′-3′) |
|---|---|---|
| Cerulean | AAGCTGACCCTGAAGTT | CTTGTAGTTGCCGTCGT |
| | CATCTGC | CCTTGAA |
| VSX2 | TCATGGCGGAGTATGGG | TCCAGCGACTTTTTGTG |
| | CT | CATC |
| mCherry | GATAACATGGCCATCAT | CGTGGCCGTTCACGGAG |
| | CAAGGA | |
| RCVRN | CCAGAGCATCTACGCCA | CCGTCGAGGTTGGAATC |
| | AGTT | GAAG |
| eGFP | GACCAAAAGATCATGGT | GAACTTCAGGGTCAGCT |
| | GAGC | TGC |
| BRN3b | CTCGCTCGAAGCCTACT | GACGCGCACCACGTTTT |
| | TTG | TC |
| GAPDH | CAATGACCCCTTCATTG | GACAAGCTTCCCGTTCT |
| | ACC | CAG |

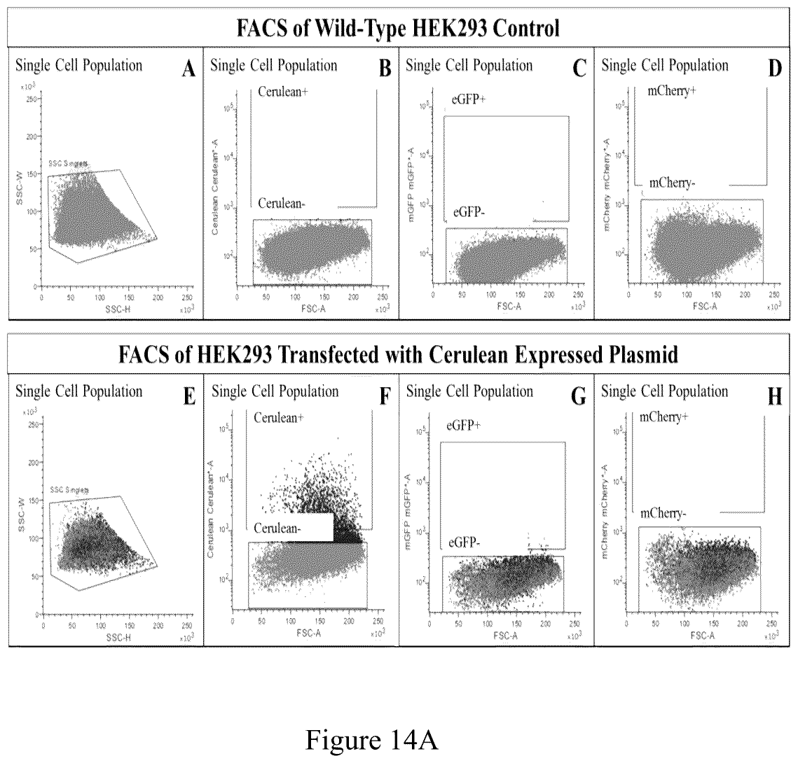

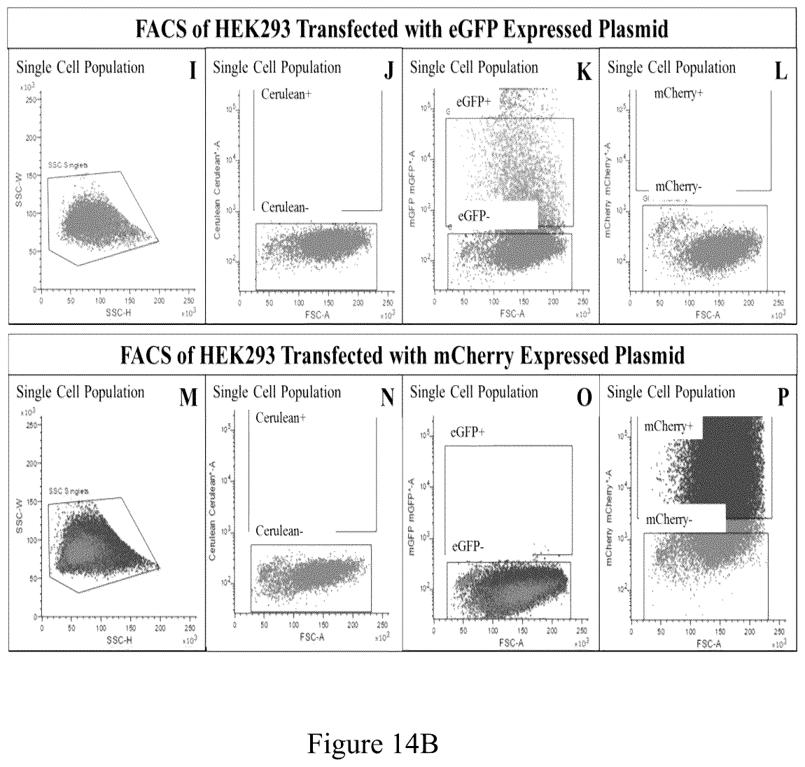

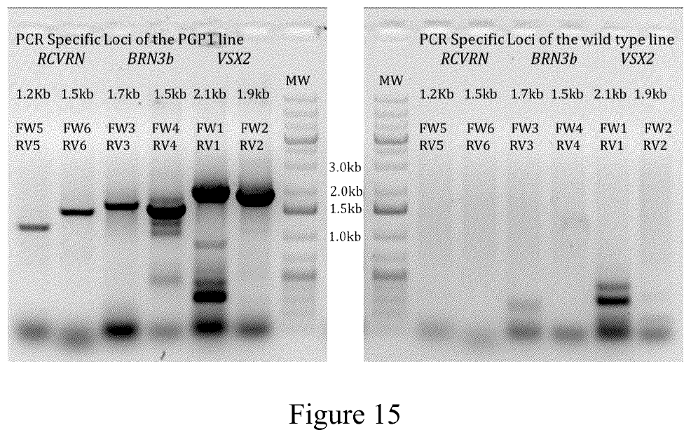

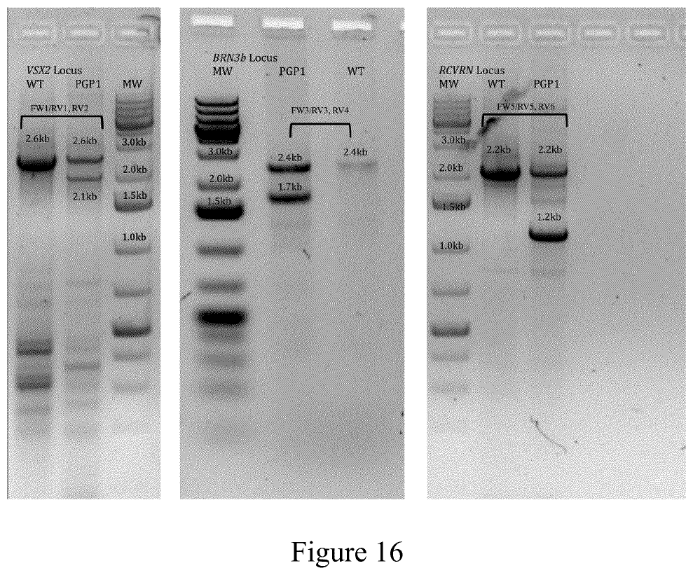

Fluorescence-Activated Cell Sorting (FACS) Analysis of the Retinal Organoids: Thirty organoids each from D55 and D135 were incubated at 37° C. in Accutase (Innovative Cell Technologies, AT104) for 20 minutes, broken down to single cells by pipetting, and filtered through a 40 μm strainer (Fishersci, 22-363-547) to eliminate cell clumps, and resuspended in ice cold 5% FBS/1×HBSS (Fishersci, 14-025-092) at a concentration of 10 million cells/ml. These cells were filtered again through a second strainer (Fisher, 08-771-23) prior to sort. For the D55 sort, single cells were sorted to separate the Cerulean positive from the Cerulean negative population, and the eGFP positive from the eGFP negative population using a FACSMelody Cell Sorter (BD Biosciences). For the D135 sort, single cells were sorted to separate the mCherry positive from the mCherry negative population. The sorted populations were used to measure mRNA transcripts for Cerulean, VSX2, mCherry, RCVRN, eGFP, BRN3b, and GAPDH via RT-qPCR using the primers listed in Table 12. Gates for sorting disaggregated retinal organoids were established in a previous experiment using HEK293 cells transiently transfected with and expression plasmid for Cerulean, eGFP, or mCherry. FACS was conducted 48 hours after transfection to determine the appropriate gates for each fluorescent protein (FIG. 14). In addition, organoids produced from wild-type hiPSCs were sorted using these established gates as a negative control.

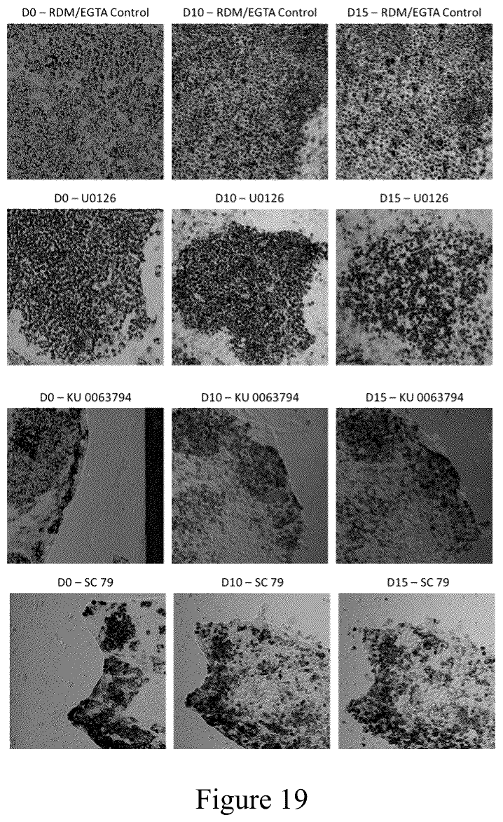

PGP1 hiPSC cells were differentiated into human retina pigment epithelium (RPE) to test the hypothesis that human RPE possesses the capacity to reprogram to neural retina (NR) if given the appropriate conditions. To test this hypothesis, Applicants have been screened a number of small molecules from two small molecule libraries. The first library consisted of 127 compounds designed to target multiple developmental pathways. Three of the compounds in this library produced blue fluorescent cells over a period of 5-15 days of treatment. These compounds were U0126 (a MEK inhibitor), SC 79 (An AKT activator), and KU 0063794 (An MTOR inhibitor). All these three compounds have all shown effectiveness in inducing Cerulean expression in at least 9 different replicates. FIG. 19 shows representative data from these three compounds. Applicants also found that the combination of SC 79 and KU 0063794 appeared to significantly synergize with respect to the induction of Cerulean expression in PGP1-derived RPE (not shown).

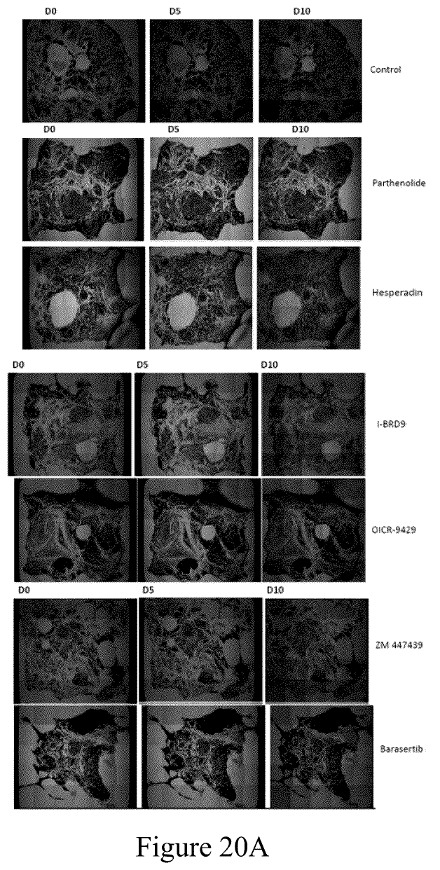

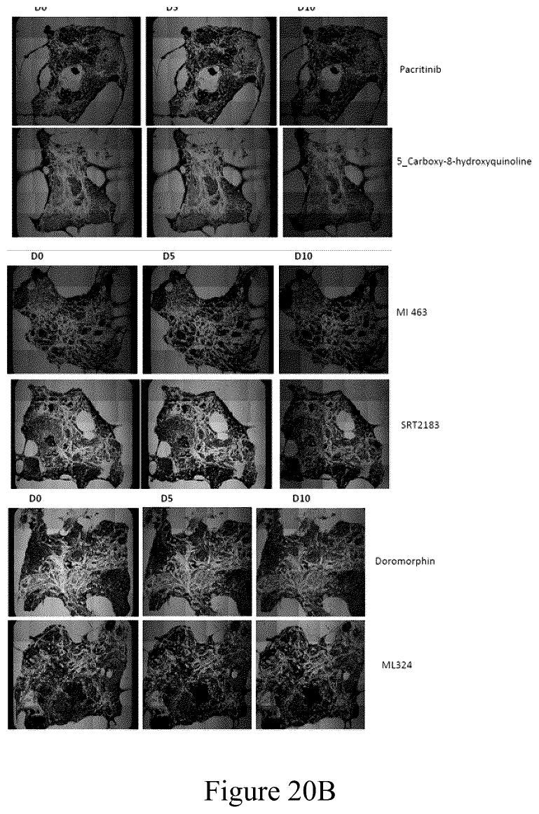

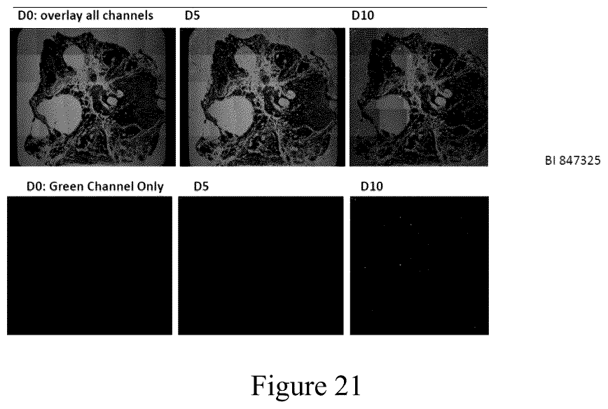

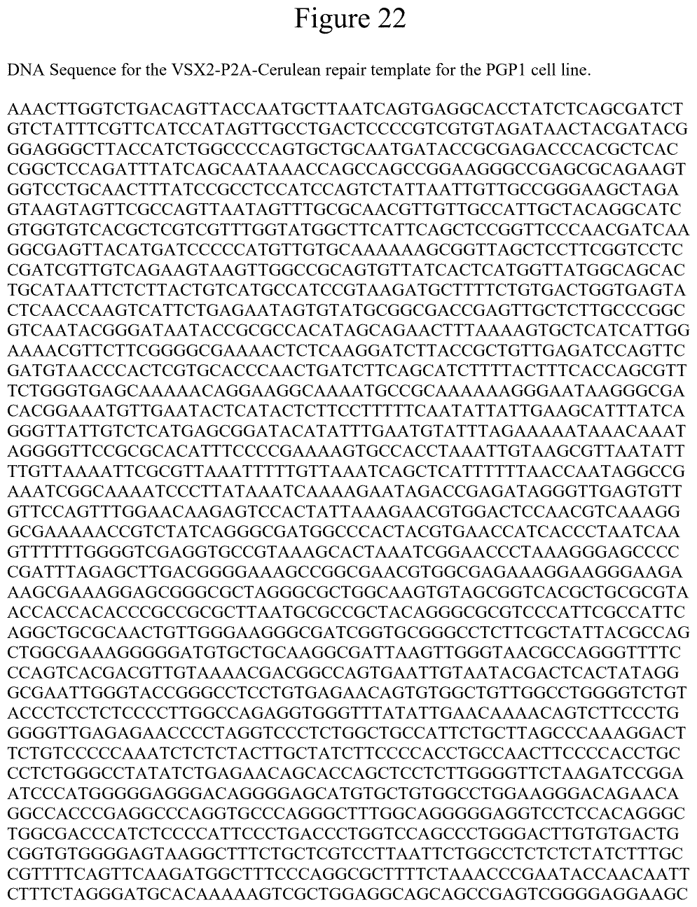

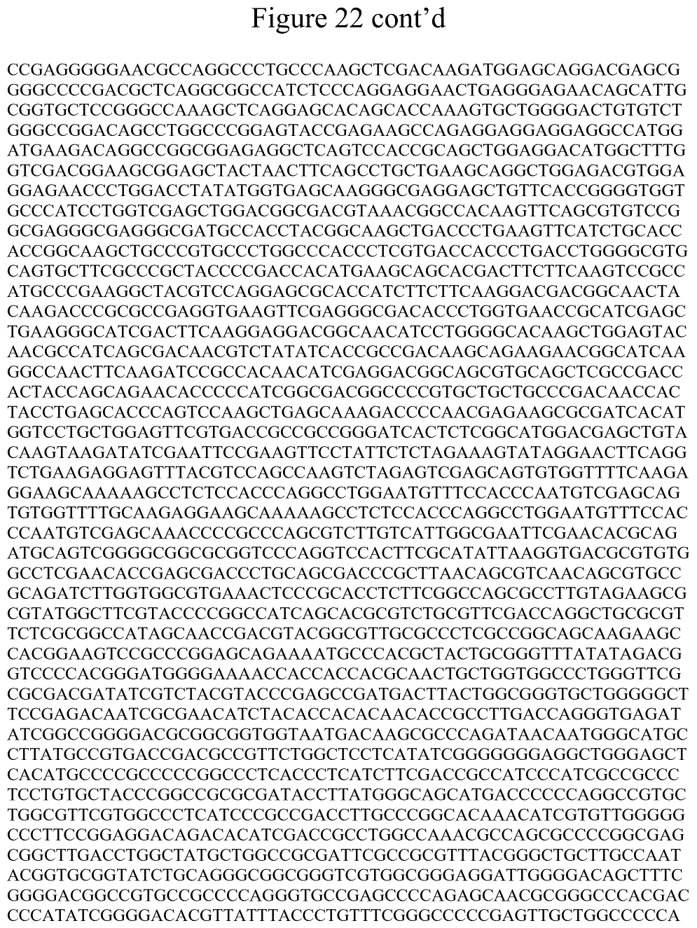

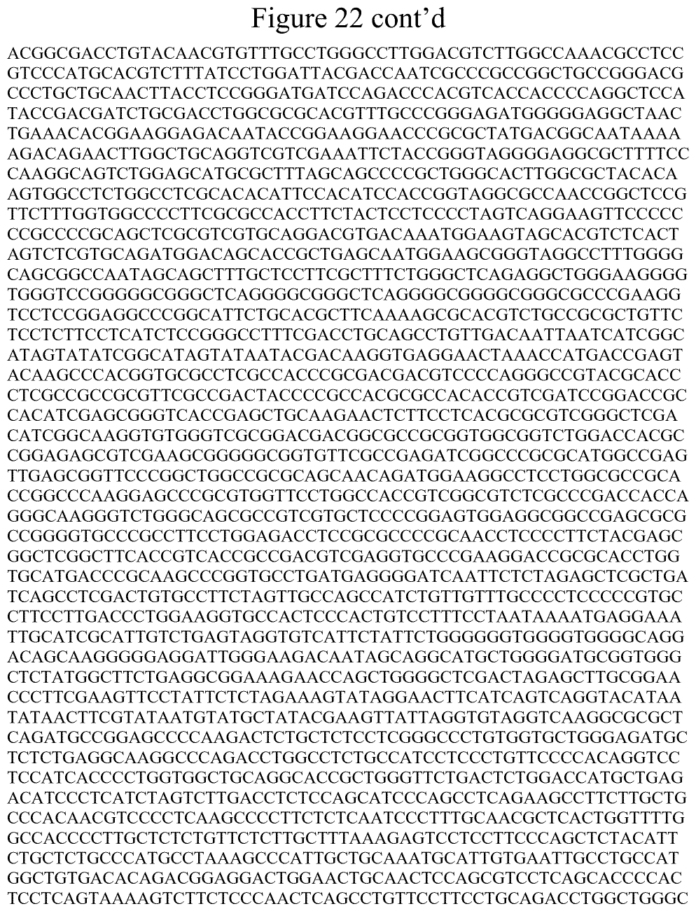

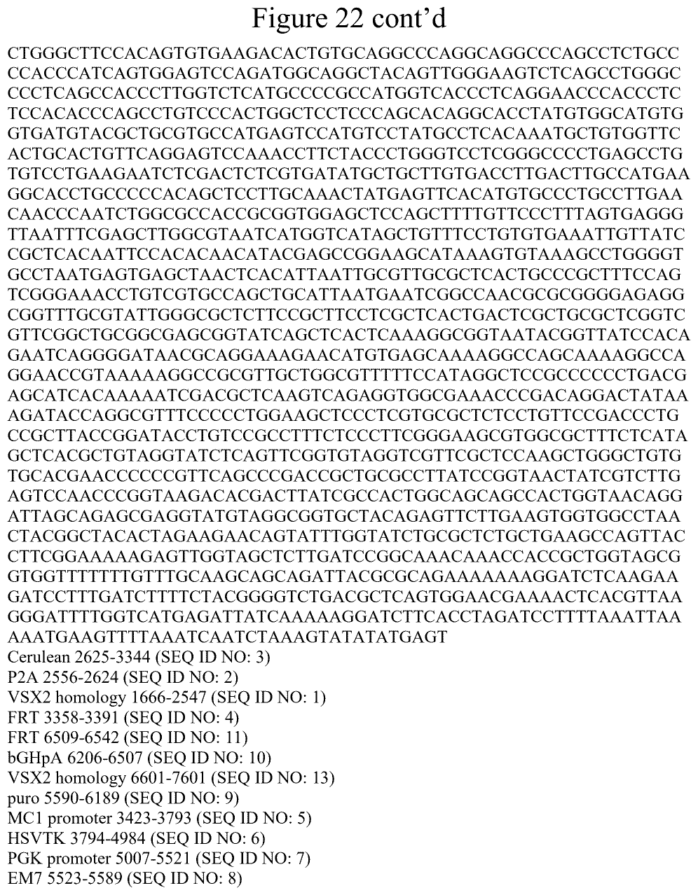

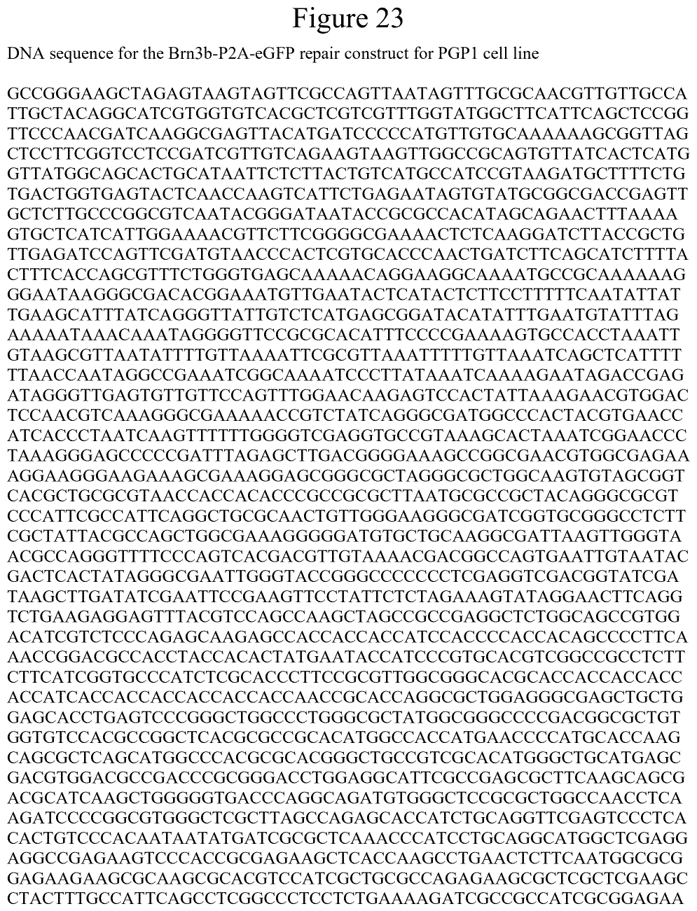

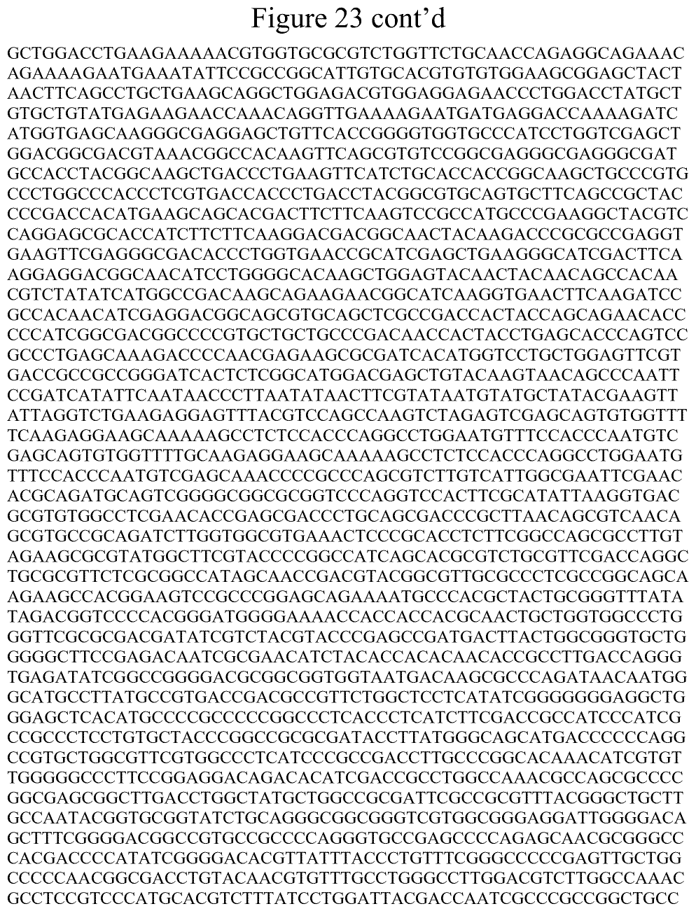

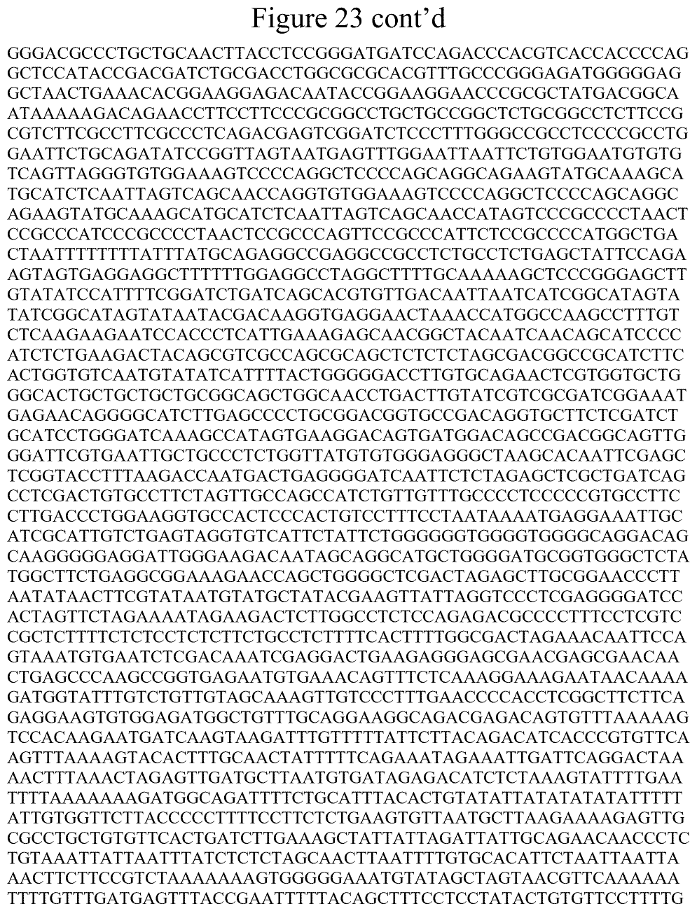

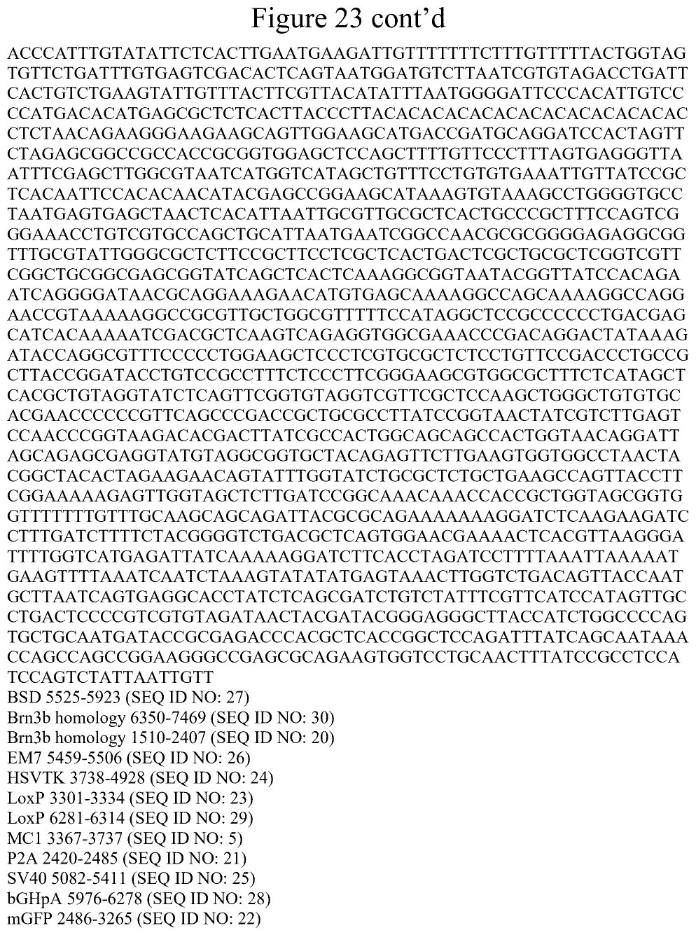

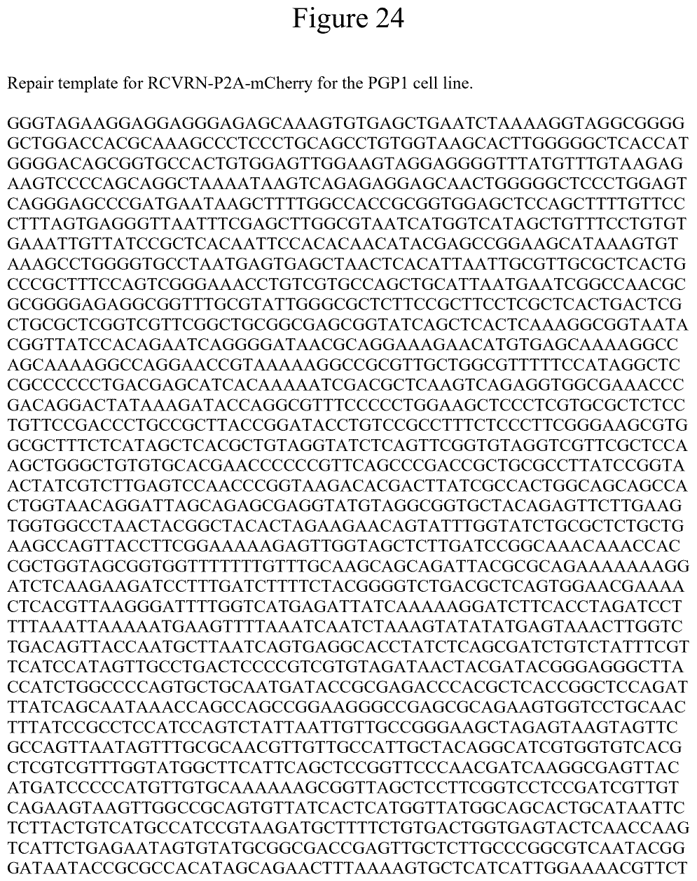

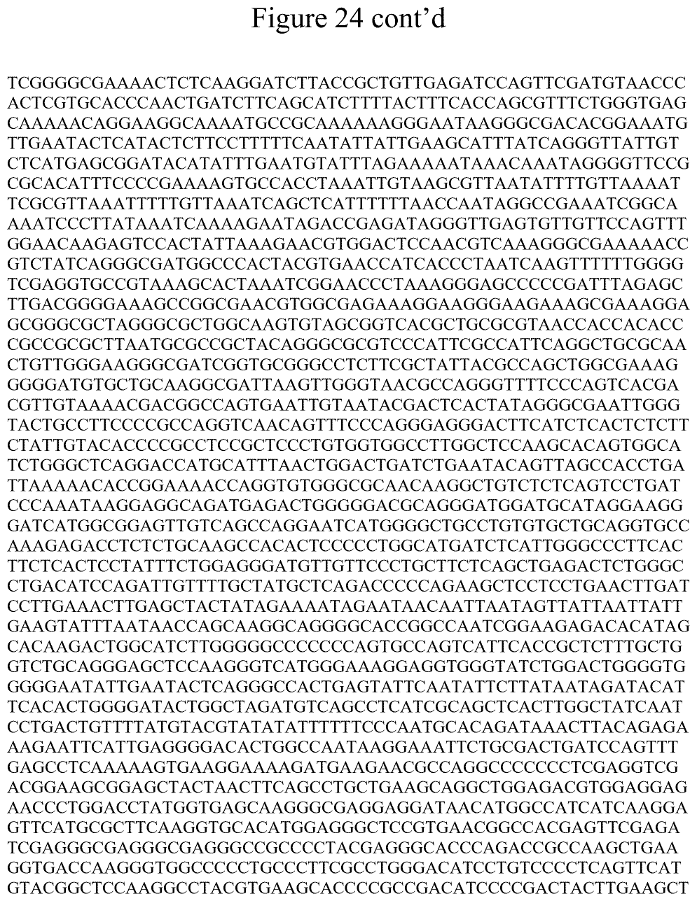

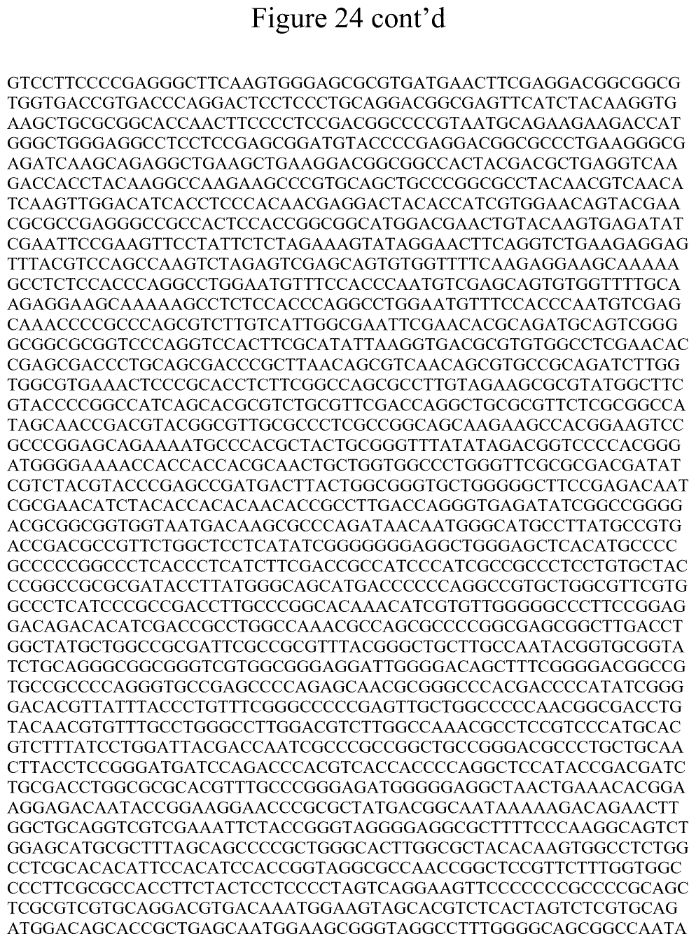

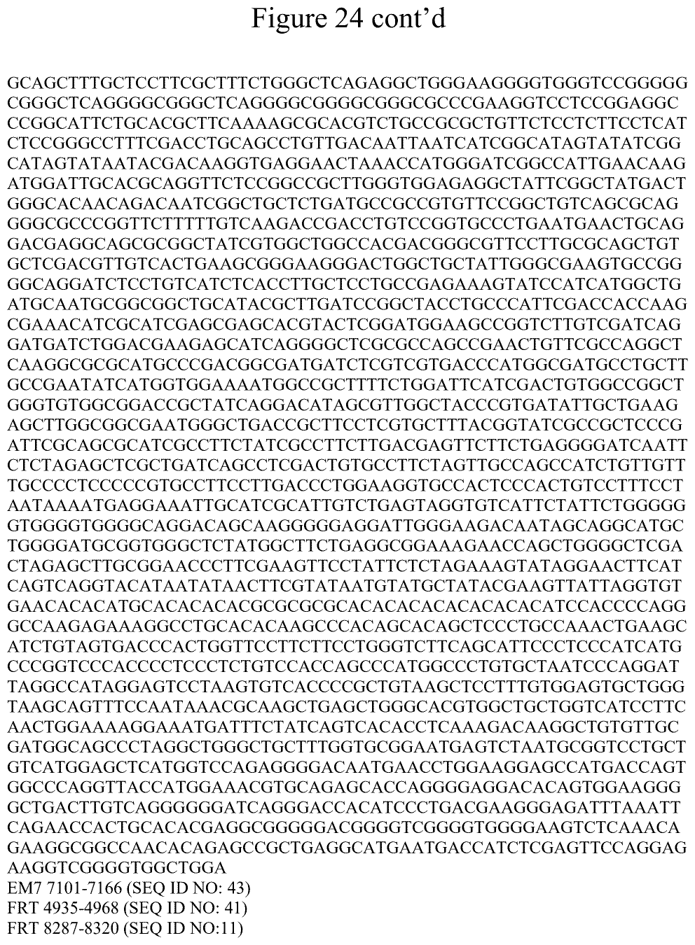

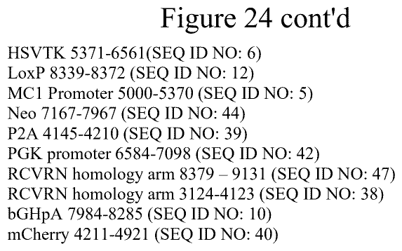

A second small molecule screen tested 180 compounds at a concentration of 40 μM designed to target epigenetic pathways (from the Selleck Chemicals Epigenetic Compounds Library L1900). Thirteen of these compounds showed evidence of inducing Cerulean expression in PGP1-derived RPE. These compounds include: Parthenolide (An NFκB inhibitor), Hesperadin (an inhibitor of Aurora Kinases A and B), OICR-9429 (An inhibitor of WDR5 interaction), I-BRD9 (An inhibitor of BRD9, a component of the SWI/SNF chromatin remodeling complex), ZM 447439 (An inhibitor of Aurora A and B Kinase), BI 847325 (An inhibitor of MEK and Aurora Kinases), Barasertib (A selective Aurora B Kinase inhibitor), Pacritinib (A JAK2 and FLT3 Kinase inhibitor), Carboxy-8-hydroxyquinoline (A histone demethylase JumonjiC JMJD2 (H3K4me3) inhibitor), MI 463 (An inhibitor of Menin-MLL interaction—decreases expression of Meis1 and HOXA9), SSRT2183 (A SIRTI1 activator similar to resveratrol), Dorsomorphin (AMP Kinase and ALK2, ALK3 and ALK6 inhibitor), ML324 (A jumonji histone demethylase (JMJD2) inhibitor H3K4). Representative images from these compounds are shown in FIG. 20. Surprisingly, BI 847325 not only induces Cerulean expression, but subsequently induces the expression of eGFP suggesting a subsequent differentiation of retinal ganglion cells from the neural retina progenitor cells (FIG. 21).

As disclosed and suggested herein, the scope of the general inventive concepts are not intended to be limited to the particular exemplary embodiments shown and described herein. From the disclosure given, those skilled in the art will not only understand the general inventive concepts and their attendant advantages but will also find apparent various changes and modifications to the methods and systems disclosed. It is sought, therefore, to cover all such changes and modifications as fall within the spirit and scope of the general inventive concepts, as described and suggested herein, and any equivalents thereof.

1. A method for the production of retinal tissue, the method comprising providing human induced pluripotent stem cells (hiPSCs), contacting the hiPSC with an expression vector to edit at least one neural retina-specific gene and culturing the transfected cell.

2. The method of claim 1, wherein the at least one neural retina-specific gene is edited at the stop codon.

3. The method of claim 1, wherein editing comprises nucleofection.

4. The method of claim 3, wherein the cell is nucleofected with at least one fluorescent reporter fusion gene.

5. The method of claim 4, wherein the at least one neural retina-specific gene is selected from VSX2, BRN3B, RCVRN, and combinations thereof.

6. The method of claim 5, wherein each of VSX2, BRN3B, RCVRN, are edited.


SEQUENCE LISTING (180 sequences)

SEQ ID NO: 1 - DNA, 883 bp, synthetic construct
  misc_feature 1..883: note = Description of Artificial Sequence: Synthetic polynucleotide
  source 1..883: mol_type = other DNA
tcctgtgagaacagtgtggctgttggcctggggtctgtaccctcctctccccttggccagaggtgggtttatattgaacaaaacagtcttccctgggggttgagagaacccctaggtccctctggctgccattctgcttagcccaaaggacttctgtcccccaaatctctctacttgctatcttccccacctgccaacttccccacctgccctctgggcctatatctgagaacagcaccagctcctcttggggttctaagatccggaatcccatgggggagggacaggggagcatgtgctgtggcctggaagggacagaacaggccacccgaggcccaggtgcccagggctttggcagggggaggtcctccacagggctggcgacccatctccccattccctgaccctggtccagccctgggacttgtgtgactgcggtgtggggagtaaggctttctgctcgtccttaattctggcctctctctatctttgccgttttcagttcaagatggctttcccaggcgcttttctaaacccgaataccaacaattctttctagggatgcacaaaaagtcgctggaggcagcagccgagtcggggaggaagcccgagggggaacgccaggccctgcccaagctcgacaagatggagcaggacgagcggggccccgacgctcaggcggccatctcccaggaggaactgagggagaacagcattgcggtgctccgggccaaagctcaggagcacagcaccaaagtgctggggactgtgtctgggccggacagcctggcccggagtaccgagaagccagaggaggaggaggccatggatgaagacaggccggcggagaggctcagtccaccgcagctggaggacatggctt

SEQ ID NO: 2 - DNA, 69 bp, synthetic construct
  misc_feature 1..69: note = Description of Artificial Sequence: Synthetic oligonucleotide
  source 1..69: mol_type = other DNA
cggaagcggagctactaacttcagcctgctgaagcaggctggagacgtggaggagaaccctggacctat

SEQ ID NO: 3 - DNA, 721 bp, synthetic construct
  misc_feature 1..721: note = Description of Artificial Sequence: Synthetic polynucleotide
  source 1..721: mol_type = other DNA
aatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 4 - DNA, 34 bp, synthetic construct
  misc_feature 1..34: note = Description of Artificial Sequence: Synthetic oligonucleotide
  source 1..34: mol_type = other DNA
gaagttcctattctctagaaagtataggaacttc

SEQ ID NO: 5 - DNA, 371 bp, synthetic construct
  misc_feature 1..371: note = Description of Artificial Sequence: Synthetic polynucleotide
  source 1..371: mol_type = other DNA
tctagagtcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgt

SEQ ID NO: 6 - DNA, 1191 bp, synthetic construct
  misc_feature 1..1191: note = Description of Artificial Sequence: Synthetic polynucleotide
  source 1..1191: mol_type = other DNA
atggcttcgtaccccggccatcagcacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgccttgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatgatccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaa

SEQ ID NO: 7 - DNA, 537 bp, synthetic construct
  misc_feature 1..537: note = Description of Artificial Sequence: Synthetic polynucleotide
  source 1..537: mol_type = other DNA
cttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagc

SEQ ID NO: 8 - DNA, 68 bp, synthetic construct
  misc_feature 1..68: note = Description of Artificial Sequence: Synthetic oligonucleotide
  source 1..68: mol_type = other DNA
ctgttgacaattaatcatcggcatagtatatcggcatagtataatacgacaaggtgaggaactaaacc

SEQ ID NO: 9 - DNA, 616 bp, synthetic construct
  misc_feature 1..616: note = Description of Artificial Sequence: Synthetic polynucleotide
  source 1..616: mol_type = other DNA
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgaggggatcaattct

SEQ ID NO: 10 - DNA, 318 bp, synthetic construct
  misc_feature 1..318: note = Description of Artificial Sequence: Synthetic polynucleotide
  source 1..318: mol_type = other DNA
tgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaaccctt

SEQ ID NO: 11 - DNA, 35 bp, synthetic construct
  misc_feature 1..35: note = Description of Artificial Sequence: Synthetic oligonucleotide
  source 1..35: mol_type = other DNA
cgaagttcctattctctagaaagtataggaacttc

SEQ ID NO: 12 - DNA, 52 bp, synthetic construct
  misc_feature 1..52: note = Description of Artificial Sequence: Synthetic oligonucleotide
  source 1..52: mol_type = other DNA
atcagtcaggtacataatataacttcgtataatgtatgctatacgaagttat

SEQ ID NO: 13 - DNA, 1001 bp, synthetic construct
  misc_feature 1..1001: note = Description of Artificial Sequence: Synthetic polynucleotide
  source 1..1001: mol_type = other DNA
taggtcaaggcgcgctcagatgccggagccccaagactctgctctcctcgggccctgtggtgctgggagatgctctctgaggcaaggcccagacctggcctctgccatcctccctgttccccacaggtcctccatcacccctggtggctgcaggcaccgctgggttctgactctggaccatgctgagacatccctcatctagtcttgacctctccagcatcccagcctcagaagccttcttgctgcccacaacgtcccctcaagccccttctctcaatccctttgcaacgctcactggttttggccaccccttgctctctgttctcttgctttaaagagtcctccttcccagctctacattctgctctgcccatgcctaaagcccattgctgcaaatgcattgtgaattgcctgccatggctgtgacacagacggaggactggaactgcaactccagcgtcctcagcaccccactcctcagtaaaagtcttctcccaactcagcctgttccttcctgcagacctggctgggcctgggcttccacagtgtgaagacactgtgcaggcccaggcaggcccagcctctgccccacccatcagtggagtccagatggcaggctacagttgggaagtctcagcctgggcccctcagccacccttggtctcatgccccgccatggtcaccctcaggaacccaccctctccacacccagcctgtcccactggctcctcccagcacaggcacctatgtggcatgtggtgatgtacgctgcgtgccatgagtccatgtcctatgcctcacaaatgctgtggttcactgcactgttcaggagtccaaaccttctaccctgggtcctcgggcccctgagcctgtgtcctgaagaatctcgactctcgtgatatgctgcttgtgaccttgacttgccatgaaggcacctgcccccacagctccttgcaaactatgagttcacatgtgccctgccttgaacaacccaatctggc

SEQ ID NO: 14 - DNA, 72 bp, synthetic construct
  misc_feature 1..72: note = Description of Artificial Sequence: Synthetic oligonucleotide
  source 1..72: mol_type = other DNA
ggatccactagttctagagcggccgccaccgcggtggagctccagcttttgttccctttagtgagggttaat

SEQ ID NO: 15 - DNA, 66 bp, synthetic construct
  misc_feature 1..66: note = Description of Artificial Sequence: Synthetic oligonucleotide
  source 1..66: mol_type = other DNA
ttcgagcttggcgtaatcatggtcatagctgtttcctgtgtgaaattgttatccgctcacaattcc

SEQ ID NO: 16 - DNA, 34 bp, synthetic construct
  misc_feature 1..34: note = Description of Artificial Sequence: Synthetic oligonucleotide
  source 1..34: mol_type = other DNA
acacaacatacgagccggaagcataaagtgtaaa

SEQ ID NO: 17 - DNA, 35 bp, synthetic construct
  misc_feature 1..35: note = Description of Artificial Sequence: Synthetic oligonucleotide
  source 1..35: mol_type = other DNA
gcctggggtgcctaatgagtgagctaactcacatt

SEQ ID NO: 18 - DNA, 1050 bp, synthetic construct
  misc_feature 1..1050: note = Description of Artificial Sequence: Synthetic polynucleotide
  source 1..1050: mol_type = other DNA
aattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtta

SEQ ID NO: 19 - DNA, 1689 bp, synthetic construct
  misc_feature 1..1689: note = Description of Artificial Sequence: Synthetic polynucleotide
  source 1..1689: mol_type = other DNA
ccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatgaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagt

SEQ ID NO: 20 - DNA, 898 bp, synthetic construct
  misc_feature 1..898: note = Description of Artificial Sequence: Synthetic polynucleotide
  source 1..898: mol_type = other DNA
cgccgaggctctggcagccgtggacatcgtctcccagagcaagagccaccaccaccatccaccccaccacagccccttcaaaccggacgccacctaccacactatgaataccatcccgtgcacgtcggccgcctcttcttcatcggtgcccatctcgcacccttccgcgttggcgggcacgcaccaccaccaccaccatcaccaccaccaccaccaccaaccgcaccaggcgctggagggcgagctgctggagcacctgagtcccgggctggccctgggcgctatggcgggccccgacggcgctgtggtgtccacgccggctcacgcgccgcacatggccaccatgaaccccatgcaccaagcagcgctcagcatggcccacgcgcacgggctgccgtcgcacatgggctgcatgagcgacgtggacgccgacccgcgggacctggaggcattcgccgagcgcttcaagcagcgacgcatcaagctgggggtgacccaggcagatgtgggctccgcgctggccaacctcaagatccccggcgtgggctcgcttagccagagcaccatctgcaggttcgagtccctcacactgtcccacaataatatgatcgcgctcaaacccatcctgcaggcatggctcgaggaggccgagaagtcccaccgcgagaagctcaccaagcctgaactcttcaatggcgcggagaagaagcgcaagcgcacgtccatcgctgcgccagagaagcgctcgctcgaagcctactttgccattcagcctcggccctcctctgaaaagatcgccgccatcgcggagaagctggacctgaagaaaaacgtggtgcgcgtctggttctgcaaccagaggcagaaacagaaaagaatgaaatattccgccggcatt

SEQ ID NO: 21 - DNA, 80 bp, synthetic construct
  misc_feature 1..80: note = Description of Artificial Sequence: Synthetic oligonucleotide
  source 1..80: mol_type = other DNA
gtgcacgtgtgtggaagcggagctactaacttcagcctgctgaagcaggctggagacgtggaggagaaccctggacctat

SEQ ID NO: 22 - DNA, 778 bp, synthetic construct
  misc_feature 1..778: note = Description of Artificial Sequence: Synthetic polynucleotide
  source 1..778: mol_type = other DNA
gctgtgctgtatgagaagaaccaaacaggttgaaaagaatgatgaggaccaaaagatcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 23 - DNA, 101 bp, synthetic construct
  misc_feature 1..101: note = Description of Artificial Sequence: Synthetic polynucleotide
  source 1..101: mol_type = other DNA
cagcccaattccgatcatattcaataacccttaatataacttcgtataatgtatgctatacgaagttattaggtctgaagaggagtttacgtccagccaag

SEQ ID NO: 24 - DNA, 1496 bp, synthetic construct
  misc_feature 1..1496: note = Description of Artificial Sequence: Synthetic polynucleotide
  source 1..1496: mol_type = other DNA
atggcttcgtaccccggccatcagcacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgccttgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatgatccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaaccttccttcccgcggcctgctgccggctctgcggcctcttccgcgtcttcgccttcgccctcagacgagtcggatctccctttgggccgcctccccgcctggaattctgcagatatccggttagtaatgagtttggaattaattctgtggaaccttccttcccgcggcctgctgccggctctgcggcctcttccgcgtcttcgccttcgccctcagacgagtcggatctccctttgggccgcctccccgcctggaattctgcagatatccggttagtaatgagtttggaattaattctgtggaat

SEQ ID NO: 25 - DNA, 330 bp, synthetic construct
  misc_feature 1..330: note = Description of Artificial Sequence: Synthetic polynucleotide
  source 1..330: mol_type = other DNA
gtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaa

SEQ ID NO: 26 - DNA, 95 bp, synthetic construct
  misc_feature 1..95: note = Description of Artificial Sequence: Synthetic oligonucleotide
  source 1..95: mol_type = other DNA
aagctcccgggagcttgtatatccattttcggatctgatcagcacgtgttgacaattaatcatcggcatagtatatcggcatagtataatacgac

SEQ ID NO: 27 - DNA, 417 bp, synthetic construct
  misc_feature 1..417: note = Description of Artificial Sequence: Synthetic polynucleotide
  source 1..417: mol_type = other DNA
aaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaa

SEQ ID NO: 28 - DNA, 355 bp, synthetic construct
  misc_feature 1..355: note = Description of Artificial Sequence: Synthetic polynucleotide
  source 1..355: mol_type = other DNA
gcacaattcgagctcggtacctttaagaccaatgactgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaaccctta

SEQ ID NO: 29 - DNA, 70 bp, synthetic construct
  misc_feature 1..70: note = Description of Artificial Sequence: Synthetic oligonucleotide
  source 1..70: mol_type = other DNA
atataacttcgtataatgtatgctatacgaagttattaggtccctcgaggggatccactagttctagaaa

SEQ ID NO: 30 - DNA, 1121 bp, synthetic construct
  misc_feature 1..1121: note = Description of Artificial Sequence: Synthetic polynucleotide
  source 1..1121: mol_type = other DNA
atagaagactcttggcctctccagagacgcccctttcctcgtccgctcttttctctcctctcttctgcctcttttcacttttggcgactagaaacaattccagtaaatgtgaatctcgacaaatcgaggactgaagagggagcgaacgagcgaacaactgagcccaagccggtgagaatgtgaaacagtttctcaaaggaaagaataacaaaagatggtatttgtctgttgtagcaaagttgtccctttgaaccccacctcggcttcttcagaggaagtgtggagatggctgtttgcaggaaggcagacgagacagtgtttaaaaagtccacaagaatgatcaagtaagatttgtttttattcttacagacatcacccgtgttcaagtttaaaagtacactttgcaactatttttcagaaatagaaattgattcaggactaaaactttaaactagagttgatgcttaatgtgatagagacatctctaaagtattttgaattttaaaaaaagatggcagattttctgcatttacactgtatattatatatatatttttattgtggttcttacccccttttccttctctgaagtgttaatgcttaagaaaagagttgcgcctgctgtgttcactgatcttgaaagctattattagattattgcagaacaaccctctgtaaattattaatttatctctctagcaacttaattttgtgcacattctaattaattaaacttcttccgtctaaaaaaagtgggggaaatgtatagctagtaacgttcaaaaaattttgtttgatgagtttaccgaatttttacagctttcctcctatactgtgttccttttgacccatttgtatattctcacttgaatgaagattgtttttttctttgtttttactggtagtgttctgatttgtgagtcgacactcagtaatggatgtcttaatcgtgtagacctgattcactgtctgaagtattgtttacttcgttacatatttaatggggattcccacattgtccccatgacacatgagcgctctcacttacccttacacacacacacacacacacacacacctctaacagaagggaagaagcagttggaagcatgaccgatgca

SEQ ID NO: 31 - DNA, 68 bp, synthetic construct
  misc_feature 1..68: note = Description of Artificial Sequence: Synthetic oligonucleotide
  source 1..68: mol_type = other DNA
ttcgagcttggcgtaatcatggtcatagctgtttcctgtgtgaaattgttatccgctcacaattccac

SEQ ID NO: 32 - DNA, 32 bp, synthetic construct
  misc_feature 1..32: note = Description of Artificial Sequence: Synthetic oligonucleotide
  source 1..32: mol_type = other DNA
acaacatacgagccggaagcataaagtgtaaa

SEQ ID NO: 33 - DNA, 914 bp, synthetic construct
  misc_feature 1..914: note = Description of Artificial Sequence: Synthetic polynucleotide
  source 1..914: mol_type = other DNA
aattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctc

SEQ ID NO: 34 - DNA, 993 bp, synthetic construct
  misc_feature 1..993: note = Description of Artificial Sequence: Synthetic polynucleotide
  source 1..993: mol_type = other DNA
agtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactca

SEQ ID NO: 35 - DNA, 106 bp, synthetic construct
  misc_feature 1..106: note = Description of Artificial Sequence: Synthetic polynucleotide
  source 1..106: mol_type = other DNA
tactcttcctttttcaatattattgaagcatttatcagggttattgtctcatgagcggatacatatttgaatgtatttagaaaaataaacaaataggggttccgcg

SEQ ID NO: 36 - DNA, 480 bp, synthetic construct
  misc_feature 1..480: note = Description of Artificial Sequence: Synthetic polynucleotide
  source 1..480: mol_type = other DNA
cacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcg

SEQ ID NO: 37 - DNA, 317 bp, synthetic construct
  misc_feature 1..317: note = Description of Artificial Sequence: Synthetic polynucleotide
  source 1..317: mol_type = other DNA
tcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattccgaagttcctattctctagaaagtataggaacttcaggtctgaagaggagtttacgtccagccaagctagc

SEQ ID NO: 38 - DNA, 1000 bp, synthetic construct
  misc_feature 1..1000: note = Description of Artificial Sequence: Synthetic polynucleotide
  source 1..1000: mol_type = other DNA
tgccttccccgccaggtcaacagtttcccagggagggacttcatctcactctcttctattgtacaccccgcctccgctccctgtggtggccttggctccaagcacagtggcatctgggctcaggaccatgcatttaactggactgatctgaatacagttagccacctgattaaaaacaccggaaaaccaggtgtgggcgcaacaaggctgtctctcagtcctgatcccaaataaggaggcagatgagactgggggacgcagggatggatgcataggaagggatcatggcggagttgtcagccaggaatcatggggctgcctgtgtgctgcaggtgccaaagagacctctctgcaagccacactccccctggcatgatctcattgggcccttcacttctcactcctatttctggagggatgttgttccctgcttctcagctgagactctgggcctgacatccagattgttttgctatgctcagacccccagaagctcctcctgaacttgatccttgaaacttgagctactatagaaaatagaataacaattaatagttattaattattgaagtatttaataaccagcaaggcaggggcaccggccaatcggaagagacacatagcacaagactggcatcttgggggcccccccagtgccagtcattcaccgctctttgctggtctgcagggagctccaagggtcatgggaaaggaggtgggtatctggactggggtgggggaatattgaatactcagggccactgagtattcaatattcttataatagatacattcacactggggatactggctagatgtcagcctcatcgcagctcacttggctatcaatcctgactgttttatgtacgtatatattttttcccaatgcacagataaacttacagagaaagaattcattgaggggacactggccaataaggaaattctgcgactgatccagtttgagcctcaaaaagtgaaggaaaagatgaagaacgcc

SEQ ID NO: 39 - DNA, 87 bp, synthetic construct
  misc_feature 1..87: note = Description of Artificial Sequence: Synthetic oligonucleotide
  source 1..87: mol_type = other DNA
aggccccccctcgaggtcgacggaagcggagctactaacttcagcctgctgaagcaggctggagacgtggaggagaaccctggacct

SEQ ID NO: 40 - DNA, 724 bp, synthetic construct
  misc_feature 1..724: note = Description of Artificial Sequence: Synthetic polynucleotide
  source 1..724: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagatatcgaattcc

SEQ ID NO: 41 - DNA, 65 bp, synthetic construct
  misc_feature 1..65: note = Description of Artificial Sequence: Synthetic oligonucleotide
  source 1..65: mol_type = other DNA
gaagttcctattctctagaaagtataggaacttcaggtctgaagaggagtttacgtccagccaag

SEQ ID NO: 42 - DNA, 539 bp, synthetic construct
  misc_feature 1..539: note = Description of Artificial Sequence: Synthetic polynucleotide
  source 1..539: mol_type = other DNA
cttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcct

SEQ ID NO: 43 - DNA, 66 bp, synthetic construct
  misc_feature 1..66: note = Description of Artificial Sequence: Synthetic oligonucleotide
  source 1..66: mol_type = other DNA
gttgacaattaatcatcggcatagtatatcggcatagtataatacgacaaggtgaggaactaaacc

SEQ ID NO: 44 - DNA, 801 bp, synthetic construct
  misc_feature 1..801: note = Description of Artificial Sequence: Synthetic polynucleotide
  source 1..801: mol_type = other DNA
atgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttc

SEQ ID NO: 45 - DNA, 53 bp, synthetic construct
  misc_feature 1..53: note = Description of Artificial Sequence: Synthetic oligonucleotide
  source 1..53: mol_type = other DNA
cgaagttcctattctctagaaagtataggaacttcatcagtcaggtacataat

SEQ ID NO: 46 - DNA, 40 bp, synthetic construct
  misc_feature 1..40: note = Description of Artificial Sequence: Synthetic oligonucleotide
  source 1..40: mol_type = other DNA
ataacttcgtataatgtatgctatacgaagttattaggtg

SEQ ID NO: 47 - DNA, 957 bp, synthetic construct
  misc_feature 1..957: note = Description of Artificial Sequence: Synthetic polynucleotide
  source 1..957: mol_type = other DNA
tgaacacacatgcacacacacgcgcgcgcacacacacacacacacatccaccccagggccaagagaaaggcctgcacacaagcccacagcacagctccctgccaaactgaagcatctgtagtgacccactggttccttcttcctgggtcttcagcattccctcccatcatgcccggtcccacccctccctctgtccaccagcccatggccctgtgctaatcccaggattaggccataggagtcctaagtgtcaccccgctgtaagctcctttgtggagtgctgggtaagcagtttccaataaacgcaagctgagctgggcacgtggctgctggtcatccttcaactggaaaaggaaatgatttctatcagtcacacctcaaagacaaggctgtgttgcgatggcagccctaggctgggctgctttggtgcggaatgagtctaatgcggtcctgctgtcatggagctcatggtccagaggggacaatgaacctggaaggagccatgaccagtggcccaggttaccatggaaacgtgcagagcaccaggggaggacacagtggaaggggctgacttgtcaggggggatcagggaccacatccctgacgaagggagatttaaattcagaaccactgcacacgaggcgggggacggggtcggggtggggaagtctcaaacagaaggcggccaacacagagccgctgaggcatgaatgaccatctcgagttccaggagaaggtcggggtggctggagggtagaaggaggagggagagcaaagtgtgagctgaatctaaaaggtaggcgggggctggaccacgcaaagccctccctgcagcctgtggtaagcacttgggggctcaccatggggacagcggtgccactgtggagttggaagtaggaggggtttatgtttgtaagagaagtccccagcaggctaaaataagtcagagaggagcaa

SEQ ID NO: 48 - DNA, 92 bp, synthetic construct
  misc_feature 1..92: note = Description of Artificial Sequence: Synthetic oligonucleotide
  source 1..92: mol_type = other DNA
ctgggggctccctggagtcagggagcccgatgaataagcttttggccaccgcggtggagctccagcttttgttccctttagtgagggttaat

SEQ ID NO: 49 - DNA, 61 bp, synthetic construct
  misc_feature 1..61: note = Description of Artificial Sequence: Synthetic oligonucleotide
  source 1..61: mol_type = other DNA
ttcgagcttggcgtaatcatggtcatagctgtttcctgtgtgaaattgttatccgctcaca

SEQ ID NO: 50 - DNA, 39 bp, synthetic construct
  misc_feature 1..39: note = Description of Artificial Sequence: Synthetic oligonucleotide
  source 1..39: mol_type = other DNA
attccacacaacatacgagccggaagcataaagtgtaaa

SEQ ID NO: 51 - DNA, 276 bp, synthetic construct
  misc_feature 1..276: note = Description of Artificial Sequence: Synthetic polynucleotide
  source 1..276: mol_type = other DNA
gcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaa

SEQ ID NO: 52 - DNA, 705 bp, synthetic construct
  misc_feature 1..705: note = Description of Artificial Sequence: Synthetic polynucleotide
  source 1..705: mol_type = other DNA
ggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttg

SEQ ID NO: 53 - DNA, 961 bp, synthetic construct
  misc_feature 1..961: note = Description of Artificial Sequence: Synthetic polynucleotide
  source 1..961: mol_type = other DNA
gtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactca

SEQ ID NO: 54 - DNA, 132 bp, synthetic construct
  misc_feature 1..132: note = Description of Artificial Sequence: Synthetic polynucleotide
  source 1..132: mol_type = other DNA
tactcttcctttttcaatattattgaagcatttatcagggttattgtctcatgagcggatacatatttgaatgtatttagaaaaataaacaaataggggttccgcgcacatttccccgaaaagtgccaccta

SEQ ID NO: 55 - DNA, 619 bp, synthetic construct
  misc_feature 1..619: note = Description of Artificial Sequence: Synthetic polynucleotide
  source 1..619: mol_type = other DNA
aattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattg

SEQ ID NO: 56 - DNA, 32 bp, synthetic construct
  misc_feature 1..32: note = Description of Artificial Sequence: Synthetic oligonucleotide
  source 1..32: mol_type = other DNA
taatacgactcactatagggcgaattgggtac

SEQ ID NO: 57 - DNA, 256 bp, synthetic construct
  misc_feature 1..256: note = Description of Artificial Sequence: Synthetic polynucleotide
  source 1..256: mol_type = other DNA
tttgctcacatgtnngagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 58 - DNA, 197 bp, synthetic construct
  misc_feature 1..197: note = Description of Artificial Sequence: Synthetic polynucleotide
  source 1..197: mol_type = other DNA
gaaacaccgctccggccactgctagcgttttagagctagaaatagcaagttaaaataaggctagtccgttatcaacttgaaaaagtggcaccgagtcggtgttttttgttttagagctagaaatagcaagttaaaataaggctagtccgtttttagcgcgtgcgccaattctgcagacaaatggctctagaggtacc

SEQ ID NO: 59 - DNA, 20 bp, synthetic construct
  misc_feature 1..20: note = Description of Artificial Sequence: Synthetic oligonucleotide
  source 1..20: mol_type = other DNA
gtcaaggcgcgctcagatgc

SEQ ID NO: 60 - DNA, 287 bp, synthetic construct
  misc_feature 1..287: note = Description of Artificial Sequence: Synthetic polynucleotide
  source 1..287: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatgg

SEQ ID NO: 61 - DNA, 158 bp, synthetic construct
  misc_feature 1..158: note = Description of Artificial Sequence: Synthetic polynucleotide
  source 1..158: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcgg

SEQ ID NO: 62 - DNA, 228 bp, synthetic construct
  misc_feature 1..228: note = Description of Artificial Sequence: Synthetic polynucleotide
  source 1..228: mol_type = other DNA
ggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttca

SEQ ID NO: 63 - DNA, 92 bp, synthetic construct
  misc_feature 1..92: note = Description of Artificial Sequence: Synthetic oligonucleotide
  source 1..92: mol_type = other DNA
gttggaccggtgccaccatggactataaggaccacgacggagactacaaggatcatgatattgattacaaagacgatgacgataagatggcc

SEQ ID NO: 64 - DNA, 45 bp, synthetic construct
  misc_feature 1..45: note = Description of Artificial Sequence: Synthetic oligonucleotide
  source 1..45: mol_type = other DNA
ccaaagaagaagcggaaggtcggtatccacggagtcccagcagcc

SEQ ID NO: 65 - DNA, 4101 bp, synthetic construct
  misc_feature 1..4101: note = Description of Artificial Sequence: Synthetic polynucleotide
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 66 - DNA, 27 bp, synthetic construct
  misc_feature 1..27: note = Description of Artificial Sequence: Synthetic oligonucleotide
  source 1..27: mol_type = other DNA
ctgggaggcgacgaattcggcagtgga

SEQ ID NO: 67 - DNA, 54 bp, synthetic construct
  misc_feature 1..54: note = Description of Artificial Sequence: Synthetic oligonucleotide
  source 1..54: mol_type = other DNA
gagggcagaggaagtctgctaacatgcggtgacgtcgaggagaatcctggccca

SEQ ID NO: 68 - DNA, 206 bp, synthetic construct
  misc_feature 1..206: note = Description of Artificial Sequence: Synthetic polynucleotide
  source 1..206: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccgaattctaactagagctcgctgatcagcctcg

SEQ ID NO: 69 - DNA, 432 bp, synthetic construct
  misc_feature 1..432: note = Description of Artificial Sequence: Synthetic polynucleotide
  source 1..432: mol_type = other DNA
actgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagt

SEQ ID NO: 70 - DNA, 737 bp, synthetic construct
  misc_feature 1..737: note = Description of Artificial Sequence: Synthetic polynucleotide
  source 1..737: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtg

SEQ ID NO: 71 - DNA, 105 bp, synthetic construct
  misc_feature 1..105: note = Description of Artificial Sequence: Synthetic polynucleotide
  source 1..105: mol_type = other DNA
cgcggaacccctatttgtttatttttctaaatacattcaaatatgtatccgctcatgagacaataaccctgataaatgcttcaataatattgaaaaaggaagagt

SEQ ID NO: 72 - DNA, 851 bp, synthetic construct
  misc_feature 1..851: note = Description of Artificial Sequence: Synthetic polynucleotide
  source 1..851: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 73 - DNA, 635 bp, synthetic construct
  misc_feature 1..635: note = Description of Artificial Sequence: Synthetic polynucleotide
  source 1..635: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggc

SEQ ID NO: 74 - DNA, 20 bp, synthetic construct
  misc_feature 1..20: note = Description of Artificial Sequence: Synthetic oligonucleotide
  source 1..20: mol_type = other DNA
aagagtcttctaaatgccgg

SEQ ID NO: 75 - DNA, 20 bp, synthetic construct
  misc_feature 1..20: note = Description of Artificial Sequence: Synthetic oligonucleotide
  source 1..20: mol_type = other DNA
agggaggacagctgaacagt

SEQ ID NO: 76 - DNA, 24 bp, synthetic construct
  misc_feature 1..24: note = Description of Artificial Sequence: Synthetic primer
  source 1..24: mol_type = other DNA
ccaagtggaggaagcgggagaagt

SEQ ID NO: 77 - DNA, 18 bp, synthetic construct
  misc_feature 1..18: note = Description of Artificial Sequence: Synthetic primer
  source 1..18: mol_type = other DNA
gcgttggctacccgtgat

SEQ ID NO: 78 - DNA, 24 bp, synthetic construct
  misc_feature 1..24: note = Description of Artificial Sequence: Synthetic primer
  source 1..24: mol_type = other DNA
tattcggcgggctggatgagagtc

SEQ ID NO: 79 - DNA, 21 bp, synthetic construct
  misc_feature 1..21: note = Description of Artificial Sequence: Synthetic primer
  source 1..21: mol_type = other DNA
tcgactagagcttgcggaacc

SEQ ID NO: 80 - DNA, 20 bp, synthetic construct
  misc_feature 1..20: note = Description of Artificial Sequence: Synthetic primer
  source 1..20: mol_type = other DNA
agctttgttgagcaccgact

SEQ ID NO: 81 - DNA, 20 bp, synthetic construct
  misc_feature 1..20: note = Description of Artificial Sequence: Synthetic primer
  source 1..20: mol_type = other DNA
tcgccttcttgacgagttct

SEQ ID NO: 82 - DNA, 17 bp, synthetic construct
  misc_feature 1..17: note = Description of Artificial Sequence: Synthetic primer
  source 1..17: mol_type = other DNA
cggcggcggtcacgaac

SEQ ID NO: 83 - DNA, 18 bp, synthetic construct
  misc_feature 1..18: note = Description of Artificial Sequence: Synthetic primer
  source 1..18: mol_type = other DNA
gccccagctccttattcc

SEQ ID NO: 84 - DNA, 21 bp, synthetic construct
  misc_feature 1..21: note = Description of Artificial Sequence: Synthetic primer
  source 1..21: mol_type = other DNA
gccgtcgccgatgggggtgtt

SEQ ID NO: 85 - DNA, 24 bp, synthetic construct
  misc_feature 1..24: note = Description of Artificial Sequence: Synthetic primer
  source 1..24: mol_type = other DNA
aaccaggccatatacagaactcaa

SEQ ID NO: 86 - DNA, 20 bp, synthetic construct
  misc_feature 1..20: note = Description of Artificial Sequence: Synthetic primer
  source 1..20: mol_type = other DNA
gttctcctccacgtctccag

SEQ ID NO: 87 - DNA, 20 bp, synthetic construct
  misc_feature 1..20: note = Description of Artificial Sequence: Synthetic primer
  source 1..20: mol_type = other DNA
tggatctggtcctctccatc

SEQ ID NO: 88 - DNA, 20 bp, synthetic construct
  misc_feature 1..20: note = Description of Artificial Sequence: Synthetic oligonucleotide
  source 1..20: mol_type = other DNA
gtcaaggcgtactcagatgc

SEQ ID NO: 89 - DNA, 20 bp, synthetic construct
  misc_feature 1..20: note = Description of Artificial Sequence: Synthetic oligonucleotide
  source 1..20: mol_type = other DNA
gtgaagaagtgctcagatgc

SEQ ID NO: 90 - DNA, 20 bp, synthetic construct
  misc_feature 1..20: note = Description of Artificial Sequence: Synthetic oligonucleotide
  source 1..20: mol_type = other DNA
gtgaagacaccctcagatgc

SEQ ID NO: 91 - DNA, 20 bp, synthetic construct
  misc_feature 1..20: note = Description of Artificial Sequence: Synthetic oligonucleotide
  source 1..20: mol_type = other DNA
gtcaaggcgtgctccgatgg

SEQ ID NO: 92 - DNA, 20 bp, synthetic construct
  misc_feature 1..20: note = Description of Artificial Sequence: Synthetic oligonucleotide
  source 1..20: mol_type = other DNA
gtcgaagcgggctccgatgc

SEQ ID NO: 93 - DNA, 20 bp, synthetic construct
  misc_feature 1..20: note = Description of Artificial Sequence: Synthetic oligonucleotide
  source 1..20: mol_type = other DNA
gtcaatgggagctctgatgc

SEQ ID NO: 94 - DNA, 20 bp, synthetic construct
  misc_feature 1..20: note = Description of Artificial Sequence: Synthetic oligonucleotide
  source 1..20: mol_type = other DNA
agcagtcttccagatgccgg

SEQ ID NO: 95 - DNA, 20 bp, synthetic construct
  misc_feature 1..20: note = Description of Artificial Sequence: Synthetic oligonucleotide
  source 1..20: mol_type = other DNA
aagctccttctaaatgccag

SEQ ID NO: 96 - DNA, 20 bp, synthetic construct
  misc_feature 1..20: note = Description of Artificial Sequence: Synthetic oligonucleotide
  source 1..20: mol_type = other DNA
taaagtcttctaaatgccaa

SEQ ID NO: 97 - DNA, 20 bp, synthetic construct
  misc_feature 1..20: note = Description of Artificial Sequence: Synthetic oligonucleotide
  source 1..20: mol_type = other DNA
aagagaattggaaatgccgg

SEQ ID NO: 98 - DNA, 20 bp, synthetic construct
  misc_feature 1..20: note = Description of Artificial Sequence: Synthetic oligonucleotide
  source 1..20: mol_type = other DNA
aagactctttgaaatgcctg

SEQ ID NO: 99 - DNA, 20 bp, synthetic construct
  misc_feature 1..20: note = Description of Artificial Sequence: Synthetic oligonucleotide
  source 1..20: mol_type = other DNA
aatagtcctccaaatgctgg

SEQ ID NO: 100 - DNA, 20 bp, synthetic construct
  misc_feature 1..20: note = Description of Artificial Sequence: Synthetic oligonucleotide
  source 1..20: mol_type = other DNA
agggaggccagctgaagagt

SEQ ID NO: 101 - DNA, 20 bp, synthetic construct
  misc_feature 1..20: note = Description of Artificial Sequence: Synthetic oligonucleotide
  source 1..20: mol_type = other DNA
ggagagggcagctgaacagt

SEQ ID NO: 102 - DNA, 20 bp, synthetic construct
  misc_feature 1..20: note = Description of Artificial Sequence: Synthetic oligonucleotide
  source 1..20: mol_type = other DNA
agagagatcagctgaacagt

SEQ ID NO: 103 - DNA, 20 bp, synthetic construct
  misc_feature 1..20: note = Description of Artificial Sequence: Synthetic oligonucleotide
  source 1..20: mol_type = other DNA
agaaaggacagctgaactgt

SEQ ID NO: 104 - DNA, 20 bp, synthetic construct
  misc_feature 1..20: note = Description of Artificial Sequence: Synthetic oligonucleotide
  source 1..20: mol_type = other DNA
agtgaggatagctggacagt

SEQ ID NO: 105 - DNA, 33 bp, synthetic construct
  misc_feature 1..33: note = Description of Artificial Sequence: Synthetic primer
  source 1..33: mol_type = other DNA
attgggtaccgggcctcctgtgagaacagtgtg

SEQ ID NO: 106 - DNA, 36 bp, synthetic construct
  misc_feature 1..36: note = Description of Artificial Sequence: Synthetic primer
  source 1..36: mol_type = other DNA
atacgaagttattaggtgtaggtcaaggcgcgctca

SEQ ID NO: 107 - DNA, 38 bp, synthetic construct
  misc_feature 1..38: note = Description of Artificial Sequence: Synthetic primer
  source 1..38: mol_type = other DNA
ctatagggcgaattgggtactgccttccccgccaggtc

SEQ ID NO: 108 - DNA, 36 bp, synthetic construct
  misc_feature 1..36: note = Description of Artificial Sequence: Synthetic primer
  source 1..36: mol_type = other DNA
atacgaagttattaggtgtgaacacacatgcacaca

SEQ ID NO: 109 - DNA, 45 bp, synthetic construct
  misc_feature 1..45: note = Description of Artificial Sequence: Synthetic primer
  source 1..45: mol_type = other DNA
ggcgaattggagctccaccgcggtggccgccgaggctctggcagc

SEQ ID NO: 110 - DNA, 37 bp, synthetic construct
  misc_feature 1..37: note = Description of Artificial Sequence: Synthetic primer
  source 1..37: mol_type = other DNA
ccactagttctagaaatagaagactcttggcctctcc

SEQ ID NO: 111 - DNA, 33 bp, synthetic construct
  misc_feature 1..33: note = Description of Artificial Sequence: Synthetic primer
  source 1..33: mol_type = other DNA
ccgcttccgtcgaccaaagccatgtcctccagc

SEQ ID NO: 112 - DNA, 36 bp, synthetic construct
  misc_feature 1..36: note = Description of Artificial Sequence: Synthetic primer
  source 1..36: mol_type = other DNA
ctccaccgcggtggcgccagattgggttgttcaagg

SEQ ID NO: 113 - DNA, 51 bp, synthetic construct
  misc_feature 1..51: note = Description of Artificial Sequence: Synthetic primer
  source 1..51: mol_type = other DNA
gtcgacctcgagggggggcctggcgttcttcatcttttccttcactttttg

SEQ ID NO: 114 - DNA, 34 bp, synthetic construct
  misc_feature 1..34: note = Description of Artificial Sequence: Synthetic primer
  source 1..34: mol_type = other DNA
ctccaccgcggtggccaaaagcttattcatcggg

SEQ ID NO: 115 - DNA, 36 bp, synthetic construct
  misc_feature 1..36: note = Description of Artificial Sequence: Synthetic primer
  source 1..36: mol_type = other DNA
atacagcacagcataggtccagggttctcctccacg

SEQ ID NO: 116 - DNA, 45 bp, synthetic construct
  misc_feature 1..45: note = Description of Artificial Sequence: Synthetic primer
  source 1..45: mol_type = other DNA
ttgatatcgaattcctgcagcccggggtgcatcggtcatgcttcc

SEQ ID NO: 117 - DNA, 20 bp, synthetic construct
  misc_feature 1..20: note = Description of Artificial Sequence: Synthetic primer
  source 1..20: mol_type = other DNA
gcaccgcatggcttctcacc

SEQ ID NO: 118 - DNA, 22 bp, synthetic construct
  misc_feature 1..22: note = Description of Artificial Sequence: Synthetic primer
  source 1..22: mol_type = other DNA
tgtggcggcctcccttcatctg

SEQ ID NO: 119 - DNA, 19 bp, synthetic construct
  misc_feature 1..19: note = Description of Artificial Sequence: Synthetic primer
  source 1..19: mol_type = other DNA
gcctggcacccttgacatt

SEQ ID NO: 120 - DNA, 24 bp, synthetic construct
  misc_feature 1..24: note = Description of Artificial Sequence: Synthetic primer
  source 1..24: mol_type = other DNA
ttgaggggctggagaaagataagt

SEQ ID NO: 121 - DNA, 24 bp, synthetic construct
  misc_feature 1..24: note = Description of Artificial Sequence: Synthetic primer
  source 1..24: mol_type = other DNA
cactgcccactacccactactaag

SEQ ID NO: 122 - DNA, 23 bp, synthetic construct
  misc_feature 1..23: note = Description of Artificial Sequence: Synthetic primer
  source 1..23: mol_type = other DNA
ggccccatcaaaataaaaccatc

SEQ ID NO: 123 - DNA, 21 bp, synthetic construct
  misc_feature 1..21: note = Description of Artificial Sequence: Synthetic primer
  source 1..21: mol_type = other DNA
cccgctcgctcgctcgctcac

SEQ ID NO: 124 - DNA, 21 bp, synthetic construct
  misc_feature 1..21: note = Description of Artificial Sequence: Synthetic primer
  source 1..21: mol_type = other DNA
agcaggcagagcatcccatcc

SEQ ID NO: 125 - DNA, 24 bp, synthetic construct
  misc_feature 1..24: note = Description of Artificial Sequence: Synthetic primer
  source 1..24: mol_type = other DNA
tggggagcagagacaaatagagaa

SEQ ID NO: 126 - DNA, 23 bp, synthetic construct
  misc_feature 1..23: note = Description of Artificial Sequence: Synthetic primer
  source 1..23: mol_type = other DNA
cgggagcaatatgggaaatggtc

SEQ ID NO: 127 - DNA, 18 bp, synthetic construct
  misc_feature 1..18: note = Description of Artificial Sequence: Synthetic primer
  source 1..18: mol_type = other DNA
tgttcccggccatttgta

SEQ ID NO: 128 - DNA, 24 bp, synthetic construct
  misc_feature 1..24: note = Description of Artificial Sequence: Synthetic primer
  source 1..24: mol_type = other DNA
aagcccactggaaaggtatgaact

SEQ ID NO: 129 - DNA, 22 bp, synthetic construct
  misc_feature 1..22: note = Description of Artificial Sequence: Synthetic primer
  source 1..22: mol_type = other DNA
agtttacgggagggaggtcagc

SEQ ID NO: 130 - DNA, 21 bp, synthetic construct
  misc_feature 1..21: note = Description of Artificial Sequence: Synthetic primer
  source 1..21: mol_type = other DNA
gggtggcggcagcttgataaa

SEQ ID NO: 131 - DNA, 24 bp, synthetic construct
  misc_feature 1..24: note = Description of Artificial Sequence: Synthetic primer
  source 1..24: mol_type = other DNA
gagcccccggaagcacaaatacag

SEQ ID NO: 132 - DNA, 22 bp, synthetic construct
  misc_feature 1..22: note = Description of Artificial Sequence: Synthetic primer
  source 1..22: mol_type = other DNA
atcttgccagcatccattatct

SEQ ID NO: 133 - DNA, 22 bp, synthetic construct
  misc_feature 1..22: note = Description of Artificial Sequence: Synthetic primer
  source 1..22: mol_type = other DNA
aatgggaaggggactgaacaaa

SEQ ID NO: 134 - DNA, 21 bp, synthetic construct
  misc_feature 1..21: note = Description of Artificial Sequence: Synthetic primer
  source 1..21: mol_type = other DNA
tggcagggagaaacagtagaa

SEQ ID NO: 135 - DNA, 21 bp, synthetic construct
  misc_feature 1..21: note = Description of Artificial Sequence: Synthetic primer
  source 1..21: mol_type = other DNA
ccccgaggatagcactgttgg

SEQ ID NO: 136 - DNA, 23 bp, synthetic construct
  misc_feature 1..23: note = Description of Artificial Sequence: Synthetic primer
  source 1..23: mol_type = other DNA
ggcaggcgtctccgttctcacac

SEQ ID NO: 137 - DNA, 24 bp, synthetic construct
  misc_feature 1..24: note = Description of Artificial Sequence: Synthetic primer
  source 1..24: mol_type = other DNA
cttcccggcaccaaatcactctac

SEQ ID NO: 138 - DNA, 24 bp, synthetic construct
  misc_feature 1..24: note = Description of Artificial Sequence: Synthetic primer
  source 1..24: mol_type = other DNA
accccttttattcgtgctctattg

SEQ ID NO: 139 - DNA, 22 bp, synthetic construct
  misc_feature 1..22: note = Description of Artificial Sequence: Synthetic primer
  source 1..22: mol_type = other DNA
tggccttttgctttttcacacc

SEQ ID NO: 140 - DNA, 19 bp, synthetic construct
  misc_feature 1..19: note = Description of Artificial Sequence: Synthetic primer
  source 1..19: mol_type = other DNA
ccgtagggggcgaggaacc

SEQ ID NO: 141 - DNA, 24 bp, synthetic construct
  misc_feature 1..24: note = Description of Artificial Sequence: Synthetic primer
  source 1..24: mol_type = other DNA
ggggcaagcttctccactattatc

SEQ ID NO: 142 - DNA, 21 bp, synthetic construct
  misc_feature 1..21: note = Description of Artificial Sequence: Synthetic primer
  source 1..21: mol_type = other DNA
gcccctcccctgcttatctgg

SEQ ID NO: 143 - DNA, 19 bp, synthetic construct
  misc_feature 1..19: note = Description of Artificial Sequence: Synthetic primer
  source 1..19: mol_type = other DNA
agtcccgcgtcctgctctc

SEQ ID NO: 144 - DNA, 22 bp, synthetic construct
  misc_feature 1..22: note = Description of Artificial Sequence: Synthetic primer
  source 1..22: mol_type = other DNA
cacccccggcgtcctttacctg

SEQ ID NO: 145 - DNA, 24 bp, synthetic construct
  misc_feature 1..24: note = Description of Artificial Sequence: Synthetic primer
  source 1..24: mol_type = other DNA
gtgaaggcggaaatacaaacagtc

SEQ ID NO: 146 - DNA, 20 bp, synthetic construct
  misc_feature 1..20: note = Description of Artificial Sequence: Synthetic primer
  source 1..20: mol_type = other DNA
gttccatcctgcggctcttc

SEQ ID NO: 147 - DNA, 20 bp, synthetic construct
  misc_feature 1..20: note = Description of Artificial Sequence: Synthetic primer
  source 1..20: mol_type = other DNA
tgtactcctcggtccctttc

SEQ ID NO: 148 - DNA, 20 bp, synthetic construct
  misc_feature 1..20: note = Description of Artificial Sequence: Synthetic primer
  source 1..20: mol_type = other DNA
cagtctggacactggctgaa

SEQ ID NO: 149 - DNA, 20 bp, synthetic construct
  misc_feature 1..20: note = Description of Artificial Sequence: Synthetic primer
  source 1..20: mol_type = other DNA
cggagtgaatcagctcggtg

SEQ ID NO: 150 - DNA, 20 bp, synthetic construct
  misc_feature 1..20: note = Description of Artificial Sequence: Synthetic primer
  source 1..20: mol_type = other DNA
ccggaagagttgtccatgtt

SEQ ID NO: 151 - DNA, 19 bp, synthetic construct
  misc_feature 1..19: note = Description of Artificial Sequence: Synthetic primer
  source 1..19: mol_type = other DNA
tcatggcggagtatgggct

SEQ ID NO: 152 - DNA, 21 bp, synthetic construct
  misc_feature 1..21: note = Description of Artificial Sequence: Synthetic primer
  source 1..21: mol_type = other DNA
gggcaagagccatcctttcaa

SEQ ID NO: 153 - DNA, 20 bp, synthetic construct
  misc_feature 1..20: note = Description of Artificial Sequence: Synthetic primer
  source 1..20: mol_type = other DNA
ctcgctcgaagcctactttg

SEQ ID NO: 154 - DNA, 21 bp, synthetic construct
  misc_feature 1..21: note = Description of Artificial Sequence: Synthetic primer
  source 1..21: mol_type = other DNA
ccagagcatctacgccaagtt

SEQ ID NO: 155 - DNA, 24 bp, synthetic construct
  misc_feature 1..24: note = Description of Artificial Sequence: Synthetic primer
  source 1..24: mol_type = other DNA
gacatgcgctggaacaagggaacc

SEQ ID NO: 156 - DNA, 21 bp, synthetic construct
  misc_feature 1..21: note = Description of Artificial Sequence: Synthetic primer
  source 1..21: mol_type = other DNA
aactgagcctaccacacaaca

SEQ ID NO: 157 - DNA, 21 bp, synthetic construct
  misc_feature 1..21: note = Description of Artificial Sequence: Synthetic primer
  source 1..21: mol_type = other DNA
tccaggttttctttccctagc

SEQ ID NO: 158 - DNA, 20 bp, synthetic construct
  misc_feature 1..20: note = Description of Artificial Sequence: Synthetic primer
  source 1..20: mol_type = other DNA
ctcgctgattaggctccaac

SEQ ID NO: 159 - DNA, 23 bp, synthetic construct
  misc_feature 1..23: note = Description of Artificial Sequence: Synthetic primer
  source 1..23: mol_type = other DNA
ccgcttatactgggctattttgc

SEQ ID NO: 160 - DNA, 20 bp, synthetic construct
  misc_feature 1..20: note = Description of Artificial Sequence: Synthetic primer
  source 1..20: mol_type = other DNA
cgactcgtgtttgttgatgg

SEQ ID NO: 161 - DNA, 21 bp, synthetic construct
  misc_feature 1..21: note = Description of Artificial Sequence: Synthetic primer
  source 1..21: mol_type = other DNA
tccagcgactttttgtgcatc

SEQ ID NO: 162 - DNA, 21 bp, synthetic construct
  misc_feature 1..21: note = Description of Artificial Sequence: Synthetic primer
  source 1..21: mol_type = other DNA
ctgttcatcgtgtggtacgtg

SEQ ID NO: 163 - DNA, 19 bp, synthetic construct
  misc_feature 1..19: note = Description of Artificial Sequence: Synthetic primer
  source 1..19: mol_type = other DNA
gacgcgcaccacgtttttc

SEQ ID NO: 164 - DNA, 21 bp, synthetic construct
  misc_feature 1..21: note = Description of Artificial Sequence: Synthetic primer
  source 1..21: mol_type = other DNA
ccgtcgaggttggaatcgaag

SEQ ID NO: 165 - DNA, 23 bp, synthetic construct
  misc_feature 1..23: note = Description of Artificial Sequence: Synthetic primer
  source 1..23: mol_type = other DNA
ccgggggacactgaggaaaggag

SEQ ID NO: 166 - DNA, 19 bp, synthetic construct
  misc_feature 1..19: note = Description of Artificial Sequence: Synthetic primer
  source 1..19: mol_type = other DNA
cggattcgacctccaagcc

SEQ ID NO: 167 - DNA, 24 bp, synthetic construct
  misc_feature 1..24: note = Description of Artificial Sequence: Synthetic primer
  source 1..24: mol_type = other DNA
aagctgaccctgaagttcatctgc

SEQ ID NO: 168 - DNA, 23 bp, synthetic construct
  misc_feature 1..23: note = Description of Artificial Sequence: Synthetic primer
  source 1..23: mol_type = other DNA
gataacatggccatcatcaagga

SEQ ID NO: 169 - DNA, 21 bp, synthetic construct
  misc_feature 1..21: note = Description of Artificial Sequence: Synthetic primer
  source 1..21: mol_type = other DNA
gaccaaaagatcatggtgagc

SEQ ID NO: 170 - DNA, 20 bp, synthetic construct
  misc_feature 1..20: note = Description of Artificial Sequence: Synthetic primer
  source 1..20: mol_type = other DNA
caatgaccccttcattgacc

SEQ ID NO: 171 - DNA, 24 bp, synthetic construct
  misc_feature 1..24: note = Description of Artificial Sequence: Synthetic primer
  source 1..24: mol_type = other DNA
cttgtagttgccgtcgtccttgaa

SEQ ID NO: 172 - DNA, 17 bp, synthetic construct
  misc_feature 1..17: note = Description of Artificial Sequence: Synthetic primer
  source 1..17: mol_type = other DNA
cgtggccgttcacggag

SEQ ID NO: 173 - DNA, 20 bp, synthetic construct
  misc_feature 1..20: note = Description of Artificial Sequence: Synthetic primer
  source 1..20: mol_type = other DNA
gaacttcagggtcagcttgc

SEQ ID NO: 174 - DNA, 20 bp, synthetic construct
  misc_feature 1..20: note = Description of Artificial Sequence: Synthetic primer
  source 1..20: mol_type = other DNA
gacaagcttcccgttctcag

SEQ ID NO: 175 - DNA, 63 bp, unidentified
  misc_feature 1..63: note = Description of Unknown: VSX2 nucleotide sequence
  source 1..63: mol_type = other DNA
tcttggggctccggcatctgagcgcgccttgacctaagccatgtcctccagctgcggtggact

SEQ ID NO: 176 - DNA, 61 bp, unidentified
  misc_feature 1..61: note = Description of Unknown: BRN3b nucleotide sequence
  source 1..61: mol_type = other DNA
agcggacgaggaaaggggcgtctctggagaggccaagagtcttctaaatgccggcggaata

SEQ ID NO: 177 - DNA, 62 bp, unidentified
  misc_feature 1..62: note = Description of Unknown: RCVRN nucleotide sequence
  source 1..62: mol_type = other DNA
gtggagggaggacagctgaacagttggcatcaggcgttcttcatcttttccttcactttttg

SEQ ID NO: 178 - DNA, 8815 bp, synthetic construct
  misc_feature 1..8815: note = Description of Artificial Sequence: Synthetic polynucleotide
  source 1..8815: mol_type = other DNA
aaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggcctcctgtgagaacagtgtggctgttggcctggggtctgtaccctcctctccccttggccagaggtgggtttatattgaacaaaacagtcttccctgggggttgagagaacccctaggtccctctggctgccattctgcttagcccaaaggacttctgtcccccaaatctctctacttgctatcttccccacctgccaacttccccacctgccctctgggcctatatctgagaacagcaccagctcctcttggggttctaagatccggaatcccatgggggagggacaggggagcatgtgctgtggcctggaagggacagaacaggccacccgaggcccaggtgcccagggctttggcagggggaggtcctccacagggctggcgacccatctccccattccctgaccctggtccagccctgggacttgtgtgactgcggtgtggggagtaaggctttctgctcgtccttaattctggcctctctctatctttgccgttttcagttcaagatggctttcccaggcgcttttctaaacccgaataccaacaattctttctagggatgcacaaaaagtcgctggaggcagcagccgagtcggggaggaagcccgagggggaacgccaggccctgcccaagctcgacaagatggagcaggacgagcggggccccgacgctcaggcggccatctcccaggaggaactgagggagaacagcattgcggtgctccgggccaaagctcaggagcacagcaccaaagtgctggggactgtgtctgggccggacagcctggcccggagtaccgagaagccagaggaggaggaggccatggatgaagacaggccggcggagaggctcagtccaccgcagctggaggacatggctttggtcgacggaagcggagctactaacttcagcctgctgaagcaggctggagacgtggaggagaaccctggacctatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagatatcgaattccgaagttcctattctctagaaagtataggaacttcaggtctgaagaggagtttacgtccagccaagtctagagtcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtatggcttcgtaccccggccatcagcacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgccttgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatgatccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaacttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttcgaagttcctattctctagaaagtataggaacttcatcagtcaggtacataatataacttcgtataatgtatgctatacgaagttattaggtgtaggtcaaggcgcgctcagatgccggagccccaagactctgctctcctcgggccctgtggtgctgggagatgctctctgaggcaaggcccagacctggcctctgccatcctccctgttccccacaggtcctccatcacccctggtggctgcaggcaccgctgggttctgactctggaccatgctgagacatccctcatctagtcttgacctctccagcatcccagcctcagaagccttcttgctgcccacaacgtcccctcaagccccttctctcaatccctttgcaacgctcactggttttggccaccccttgctctctgttctcttgctttaaagagtcctccttcccagctctacattctgctctgcccatgcctaaagcccattgctgcaaatgcattgtgaattgcctgccatggctgtgacacagacggaggactggaactgcaactccagcgtcctcagcaccccactcctcagtaaaagtcttctcccaactcagcctgttccttcctgcagacctggctgggcctgggcttccacagtgtgaagacactgtgcaggcccaggcaggcccagcctctgccccacccatcagtggagtccagatggcaggctacagttgggaagtctcagcctgggcccctcagccacccttggtctcatgccccgccatggtcaccctcaggaacccaccctctccacacccagcctgtcccactggctcctcccagcacaggcacctatgtggcatgtggtgatgtacgctgcgtgccatgagtccatgtcctatgcctcacaaatgctgtggttcactgcactgttcaggagtccaaaccttctaccctgggtcctcgggcccctgagcctgtgtcctgaagaatctcgactctcgtgatatgctgcttgtgaccttgacttgccatgaaggcacctgcccccacagctccttgcaaactatgagttcacatgtgccctgccttgaacaacccaatctggcgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagt

SEQ ID NO: 179 - DNA, 8977 bp, synthetic construct
  misc_feature 1..8977: note = Description of Artificial Sequence: Synthetic polynucleotide
  source 1..8977: mol_type = other DNA
gccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattccgaagttcctattctctagaaagtataggaacttcaggtctgaagaggagtttacgtccagccaagctagccgccgaggctctggcagccgtggacatcgtctcccagagcaagagccaccaccaccatccaccccaccacagccccttcaaaccggacgccacctaccacactatgaataccatcccgtgcacgtcggccgcctcttcttcatcggtgcccatctcgcacccttccgcgttggcgggcacgcaccaccaccaccaccatcaccaccaccaccaccaccaaccgcaccaggcgctggagggcgagctgctggagcacctgagtcccgggctggccctgggcgctatggcgggccccgacggcgctgtggtgtccacgccggctcacgcgccgcacatggccaccatgaaccccatgcaccaagcagcgctcagcatggcccacgcgcacgggctgccgtcgcacatgggctgcatgagcgacgtggacgccgacccgcgggacctggaggcattcgccgagcgcttcaagcagcgacgcatcaagctgggggtgacccaggcagatgtgggctccgcgctggccaacctcaagatccccggcgtgggctcgcttagccagagcaccatctgcaggttcgagtccctcacactgtcccacaataatatgatcgcgctcaaacccatcctgcaggcatggctcgaggaggccgagaagtcccaccgcgagaagctcaccaagcctgaactcttcaatggcgcggagaagaagcgcaagcgcacgtccatcgctgcgccagagaagcgctcgctcgaagcctactttgccattcagcctcggccctcctctgaaaagatcgccgccatcgcggagaagctggacctgaagaaaaacgtggtgcgcgtctggttctgcaaccagaggcagaaacagaaaagaatgaaatattccgccggcattgtgcacgtgtgtggaagcggagctactaacttcagcctgctgaagcaggctggagacgtggaggagaaccctggacctatgctgtgctgtatgagaagaaccaaacaggttgaaaagaatgatgaggaccaaaagatcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacagcccaattccgatcatattcaataacccttaatataacttcgtataatgtatgctatacgaagttattaggtctgaagaggagtttacgtccagccaagtctagagtcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtatggcttcgtaccccggccatcagcacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgccttgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatgatccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaaccttccttcccgcggcctgctgccggctctgcggcctcttccgcgtcttcgccttcgccctcagacgagtcggatctccctttgggccgcctccccgcctggaattctgcagatatccggttagtaatgagtttggaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcggtacctttaagaccaatgactgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttaatataacttcgtataatgtatgctatacgaagttattaggtccctcgaggggatccactagttctagaaaatagaagactcttggcctctccagagacgcccctttcctcgtccgctcttttctctcctctcttctgcctcttttcacttttggcgactagaaacaattccagtaaatgtgaatctcgacaaatcgaggactgaagagggagcgaacgagcgaacaactgagcccaagccggtgagaatgtgaaacagtttctcaaaggaaagaataacaaaagatggtatttgtctgttgtagcaaagttgtccctttgaaccccacctcggcttcttcagaggaagtgtggagatggctgtttgcaggaaggcagacgagacagtgtttaaaaagtccacaagaatgatcaagtaagatttgtttttattcttacagacatcacccgtgttcaagtttaaaagtacactttgcaactatttttcagaaatagaaattgattcaggactaaaactttaaactagagttgatgcttaatgtgatagagacatctctaaagtattttgaattttaaaaaaagatggcagattttctgcatttacactgtatattatatatatatttttattgtggttcttacccccttttccttctctgaagtgttaatgcttaagaaaagagttgcgcctgctgtgttcactgatcttgaaagctattattagattattgcagaacaaccctctgtaaattattaatttatctctctagcaacttaattttgtgcacattctaattaattaaacttcttccgtctaaaaaaagtgggggaaatgtatagctagtaacgttcaaaaaattttgtttgatgagtttaccgaatttttacagctttcctcctatactgtgttccttttgacccatttgtatattctcacttgaatgaagattgtttttttctttgtttttactggtagtgttctgatttgtgagtcgacactcagtaatggatgtcttaatcgtgtagacctgattcactgtctgaagtattgtttacttcgttacatatttaatggggattcccacattgtccccatgacacatgagcgctctcacttacccttacacacacacacacacacacacacacctctaacagaagggaagaagcagttggaagcatgaccgatgcaggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgtt

SEQ ID NO: 180 - DNA, 9129 bp, synthetic construct
  misc_feature 1..9129: note = Description of Artificial Sequence: Synthetic polynucleotide
  source 1..9129: mol_type = other DNA
gggtagaaggaggagggagagcaaagtgtgagctgaatctaaaaggtaggcgggggctggaccacgcaaagccctccctgcagcctgtggtaagcacttgggggctcaccatggggacagcggtgccactgtggagttggaagtaggaggggtttatgtttgtaagagaagtccccagcaggctaaaataagtcagagaggagcaactgggggctccctggagtcagggagcccgatgaataagcttttggccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtactgccttccccgccaggtcaacagtttcccagggagggacttcatctcactctcttctattgtacaccccgcctccgctccctgtggtggccttggctccaagcacagtggcatctgggctcaggaccatgcatttaactggactgatctgaatacagttagccacctgattaaaaacaccggaaaaccaggtgtgggcgcaacaaggctgtctctcagtcctgatcccaaataaggaggcagatgagactgggggacgcagggatggatgcataggaagggatcatggcggagttgtcagccaggaatcatggggctgcctgtgtgctgcaggtgccaaagagacctctctgcaagccacactccccctggcatgatctcattgggcccttcacttctcactcctatttctggagggatgttgttccctgcttctcagctgagactctgggcctgacatccagattgttttgctatgctcagacccccagaagctcctcctgaacttgatccttgaaacttgagctactatagaaaatagaataacaattaatagttattaattattgaagtatttaataaccagcaaggcaggggcaccggccaatcggaagagacacatagcacaagactggcatcttgggggcccccccagtgccagtcattcaccgctctttgctggtctgcagggagctccaagggtcatgggaaaggaggtgggtatctggactggggtgggggaatattgaatactcagggccactgagtattcaatattcttataatagatacattcacactggggatactggctagatgtcagcctcatcgcagctcacttggctatcaatcctgactgttttatgtacgtatatattttttcccaatgcacagataaacttacagagaaagaattcattgaggggacactggccaataaggaaattctgcgactgatccagtttgagcctcaaaaagtgaaggaaaagatgaagaacgccaggccccccctcgaggtcgacggaagcggagctactaacttcagcctgctgaagcaggctggagacgtggaggagaaccctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgaactgtacaagtgagatatcgaattccgaagttcctattctctagaaagtataggaacttcaggtctgaagaggagtttacgtccagccaagtctagagtcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtatggcttcgtaccccggccatcagcacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgccttgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatgatccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaacttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttcgaagttcctattctctagaaagtataggaacttcatcagtcaggtacataatataacttcgtataatgtatgctatacgaagttattaggtgtgaacacacatgcacacacacgcgcgcgcacacacacacacacacatccaccccagggccaagagaaaggcctgcacacaagcccacagcacagctccctgccaaactgaagcatctgtagtgacccactggttccttcttcctgggtcttcagcattccctcccatcatgcccggtcccacccctccctctgtccaccagcccatggccctgtgctaatcccaggattaggccataggagtcctaagtgtcaccccgctgtaagctcctttgtggagtgctgggtaagcagtttccaataaacgcaagctgagctgggcacgtggctgctggtcatccttcaactggaaaaggaaatgatttctatcagtcacacctcaaagacaaggctgtgttgcgatggcagccctaggctgggctgctttggtgcggaatgagtctaatgcggtcctgctgtcatggagctcatggtccagaggggacaatgaacctggaaggagccatgaccagtggcccaggttaccatggaaacgtgcagagcaccaggggaggacacagtggaaggggctgacttgtcaggggggatcagggaccacatccctgacgaagggagatttaaattcagaaccactgcacacgaggcgggggacggggtcggggtggggaagtctcaaacagaaggcggccaacacagagccgctgaggcatgaatgaccatctcgagttccaggagaaggtcggggtggctgga